(19) United States
(12) Patent Application Publication
(10) Pub. No.: US 2016/0252515 A1
(43) Pub. Date: Sep. 1, 2016

(54) PERSONAL GLUCOSE METERS FOR DETECTION AND QUANTIFICATION OF ENZYMES AND METABOLITES BASED ON COENZYME DETECTION

(71) Applicant: The Board of Trustees of the University of Illnois, Urbana, IL (US)

(72) Inventors: Yi Lu, Champaign, IL (US); Yu Xiang, Urbana, IL (US); JingJing ZHANG, Champaign, IL (US)

(73) Assignee: The Board of Trustees of the University of Illinois, Urbana, IL (US)

(21) Appl. No.: 15/034,780

(22) PCT Filed: Nov. 6, 2014
(86) PCT No.: PCT/US14/64314

§ 371 Date: May 5, 2016

Related U.S. Application Data
(60) Provisional application No. 61/901,688, filed on Nov. 8, 2013.

(51) Int. Cl. G01N 33/66; G01N 33/53; G01N 33/573

(52) U.S. Cl. CPC G01N 33/66; G01N 33/5735; G01N 33/5302

(57) ABSTRACT

A general methodology for highly sensitive and selective sensors and devices that can achieve portable, low-cost and quantitative detection of target enzymes and metabolites using a personal glucose meter (PGM) is disclosed. The method and sensors take advantage of the ability of PGMs to detect enzyme cofactors or coenzymes, such as nicotinamide adenine dinucleotide (NADH) or nicotinamide adenine dinucleotide phosphate (NADPH). Based on this observation, enzymes and metabolites involved in enzymatic reactions that consume or generate a coenzyme such as NADH or NADPH can be detected using PGMs, for example by measuring increases or decreases in NADH or NADPH levels. Methods of using such sensors and fluidic devices for detecting target enzymes and metabolites, for example to diagnose disease, are also provided.

### CROSS REFERENCE TO RELATED APPLICATION

This application claims priority to U.S. Application No. 61/901,688 filed Nov. 8, 2013, herein incorporated by reference.

### FIELD

This application relates to methods that take advantage of the observation that NADH and NADPH (such as that present in human serum) display a similar response as glucose to personal glucose meters (PGMs). Thus, the application provides methods to detect of a broad array of enzymes and metabolites, which can be used in combination with PGMs, as well as sensors, devices, and kits that can be used with such methods.

### BACKGROUND

The deficiency of metabolism-related enzymes in human fluids is the cause of many human diseases and abnormalites.¹ For example, glucose-6-phosphate dehydrogenase (G6PD) deficiency^{2,3} and pyruvate kinase (PK) deficiency⁴ are the first and second most common metabolite disorders caused by the deficiency of G6PD and PK in human red blood cells. The low activities or even absence of these two enzymes fail to complete the glucose metabolism and result in the accumulation of intermediates such as glucose-6-phosphate and phosphoenolpyruvate in high concentrations, which lead to a variety of symptoms including hemolysis, jaundice and even kernicterus.²⁻⁴ In addition to enzyme deficiency, the lack or accumulation of small molecular metabolites can also indicate the development of diseases and abnormalities. For instance, the low concentration of citrate in urine and high concentration of lactate in serum indicate the risk of kidney stone formation⁵⁻⁷ and lactic acidosis,⁸ respectively. In addition, lactic acidosis is also the most serious potential adverse effect of drug therapies such as the widely prescribed biguanides (e.g., metformin) for diabetes.⁹

To minimize the hazard of these diseases and ensure timely diagnosis as well as safe drug therapies, traditional methods that are currently carried out in research labs and hospitals mostly utilize the formation or consumption of nicotinamide adenine dinucleotide (NAD⁺ and NADH as the oxidized and reduced forms, respectively) in the presence of the target enzymes and metabolites,¹⁰⁻¹⁴ such as the fluorescent spot tests.^{15,16} By measuring the absorbance or fluorescence of NADH produced or consumed, the enzyme activities and metabolite concentrations can then be determined. Despite of their wide application, these methods still have limitations. First, there are still few low-cost commercial portable devices widely available for point-of-care diagnosis using the NADH-based principles, and the instrument-free tests based on eye observations are only semi-quantitative. Second, the traditional methods based on optical signals are severely interfered with by colored species such as hemoglobin in human blood, so that complicated procedures are then required to remove hemoglobin from samples before measurement, making point-of-care applications difficult.

The personal glucose meter (PGM) is currently the most successfully commercialized portable device for medical diagnosis. Its large market of more than one billion US dollars annually and technical development over tens of years makes PGM extremely low-cost, portable, widely available worldwide, and simple to use.¹⁷⁻¹⁹ The recent advancement in commercialization of mobile phone integrable PGMs further extends the spawn of users and convenience of public usage.²⁰ However, currently off-the-shelf PGMs are only used to monitor blood glucose for diabetes.

Previous studies from the inventors' group have successfully extended the range of targets that PGMs can detect to metal ions, small organic molecules and biomolecules using DNAs²¹⁻²³ and antibodies²⁴ to recognize the targets and utilized invertase to convert target recognition into glucose signals. Other scientists and researchers have also demonstrated novel approaches using PGMs to detect a variety of targets based on organic molecules,²⁵ DNAs^{26,27} and antibodies.²⁸ Unfortunately, these methods are not suitable for the detection of enzymes and metabolites that are difficult to develop DNA or antibody binders to recognize. Phillips and coworkers recently reported a method using glucose derivatives as the substrates of enzymes for enzyme activities.²⁹ However, the range of detectable enzymes by this method was limited to those using glucose derivatives as their substrates.

### SUMMARY

The present application discloses methods, sensors, and devices that can be used to detect one or more target enzymes or metabolites that are part of an enzymatic reaction that consumes or generates enzyme cofactor(s) or coenzyme(s). For example, the methods, sensors, and devices can be used to diagnose a disease or condition associated with the presence or reduction of the target.

Provided herein is a new methodology to use PGMs to detect enzymes and metabolites for point-of-care diagnosis. In some examples, such detection is indirect, for example by detection of a coenzyme, whose amount is proportional or inversely proportional to the target. The principle is based on the fact that PGMs can detect sub-mM levels of coenzymes, such as NADH or NADPH, similar to glucose, because cofactors and coenzymes, such as NADH or NADPH, are active electron donors for the electrodes of PGMs.¹⁷⁻¹⁹ For example, NADH is a coenzyme involved in many metabolism-related enzymatic reactions. Upon the conversion of a target enzyme or metabolites into NADH, the concentration of NADH changes, thus permitting a determination of the enzyme and metabolite concentrations using PGMs. Because the normal concentrations of NADH in most human fluids are much lower than mM levels, little interference occurs and the method has been used for the analysis of human serum. In addition, colored species present in blood, such as hemoglobin, do not affect the PGM measurement so that no separation procedure is required. NADH is dramatically different in chemical properties from glucose, and background concentrations of glucose in the samples can be removed using hexokinase³⁰. By this approach, the effects of varying original glucose concentrations in different samples are eliminated. Based on the observations made by detecting NADH or NADPH, this disclosure provides similar methods for detecting other coenzymes, such as the reduced form of flavin adenine dinucleotide (FADH₂), and the reduced form of flavin mononucleotide (FMNH₂), for detection of additional enzymatic pathways.

In one example the method is for detecting a target, such as a target enzyme or target metabolite. The target enzyme or target metabolite is part of an enzymatic reaction that consumes or generates a coenzyme, such as nicotinamide adenine dinucleotide (NADH), nicotinamide adenine dinucleotide phosphate (NADPH), the reduced form of flavin adenine dinucleotide (FADH₂), the reduced form of flavin mononucleotide (FMNH₂) or combinations thereof (or a functional derivative thereof). FAD and FMN are redox cofactors similar to NAD, and FADH₂ and FMNH₂ are coenzymes that can be detected using the disclosed methods. Exemplary targets include but are not limited to: glucose-6-phosphate dehydrogenase (G6PD), pyruvate kinase (PK), citrate, lactate, ethanol, 3-β-hydroxybutyrate (3HB), pyruvate, adenosine diphosphate (ADP), adenosine triphosphate (ATP), and creatinine, as well as those provided in Table 1. Such methods can also be used to determine that the subject from whom the sample was obtained has a particular disease, such as G6PD deficiency, PK deficiency, lactic acidosis and ethanol poisoning, as well as those provided in Table 1.

The method can include contacting a test sample with one or more starting products. The starting products are those reagents needed for an enzymatic reaction that utilizes the target enzyme or target metabolite and consumes or generates the enzyme cofactor or coenzyme. The starting products used will depend on the target. For example, if the target is G6PD, the starting reagents can include G6P, and NAD⁺ or NADP⁺. The sample is incubated with the starting products under conditions for the enzymatic reaction to consume or generate the coenzyme. The coenzyme remaining after this reaction is detected with a glucose meter, such as a personal glucose meter (PGM). The target enzyme or metabolite is detected by correlating the amount of coenzyme detected, for example wherein an amount of coenzyme detected is proportional or inversely proportional to an amount of target in the sample. In some examples the method also includes contacting the test sample with hexokinase, MgCl₂, and ATP under conditions sufficient to remove glucose in the test sample. In some examples, the method includes comparing a detected value or amount of coenzyme in the test sample to a reference value or range of values for the target enzyme or metabolite expected in a normal subject (or values observed for a normal test sample), to determine whether the target enzyme or metabolite in the sample is increased or decreased relative to a normal sample/subject.

In one example the disclosed methods can be used to detect one or more targets, such as at least 2, at least 3, at least 5, at least 10, or at least 20 different target enzymes or metabolites in a sample, such as 2, 3, 4, 5, 6, 7, 8, 9, 10, 15, 20, 25, 30, 40, 50, 75 or 100 different targets. In one example the disclosed methods can be used to detect one or more targets, in a plurality of samples simultaneously or contemporaneously, for example as at least 2, at least 3, at least 5, at least 10, at least 20, at least 100, or at least 200 different samples, such as 2, 3, 4, 5, 6, 7, 8, 9, 10, 15, 20, 25, 30, 40, 50, 75, 100, 200, 500, or 1000 different samples.

Also provided herein are sensors and devices for detecting target enzymes or metabolites, for example using the methods provided herein. In one example a sensor is part of a lateral flow device or fluidic device (e.g., microfluidic device or macrofluidic device). For example, the sensor in one example includes a solid support to which is attached one or more starting products needed for an enzymatic reaction that utilizes the target enzyme or target metabolite and consumes or generates the coenzyme. For example, the sensor can include a reagent pad/membrane or holding chamber containing the starting products (such as enzyme(s), substrates(s), ATP, ADP, MgCl₂, or combinations thereof). In another example, a fluidic device includes one or more mixing chambers, such as a first chamber that includes or holds buffer reagents and reagents needed for pre-treatment of the sample (e.g., hexokinase) and a second chamber that includes or holds one or more starting products needed for the enzymatic reaction that utilizes the target enzyme or target metabolite and consumes or generates the coenzyme (e.g., NAD⁺, ATP, ADP). In some examples, the sensor includes a solid support attached thereto the one or more starting products needed for an enzymatic reaction that utilizes the target enzyme or target metabolite and consumes or generates the coenzyme. For example, beads having attached thereto the one or more starting products needed for the enzymatic reaction that utilizes the target enzyme or target metabolite and consumes or generates the coenzyme can be present in one or more mixing chambers of a fluidic device, or the walls of one or more mixing chambers of the device can have attached thereto one or more starting products needed for the enzymatic reaction that utilizes the target enzyme or target metabolite and consumes or generates the coenzyme.

Also provided are kits that include the disclosed sensors, lateral flow devices, or fluidic devices. Methods of using these sensors, fluidic devices (e.g., microfluidic devices, macrofluidic devices), and lateral flow devices to detect one or more target agents, for example to diagnose a disease, are provided herein.

The foregoing and other objects and features of the disclosure will become more apparent from the following detailed description, which proceeds with reference to the accompanying figures.

### BRIEF DESCRIPTION OF THE DRAWINGS

FIGS. 1A-1D are schematic drawings showing the enzymatic reactions inducing NADH concentration changes for PGM measurement of (A) glucose-6-phosphate (G6PD), (B) PK, (C) citrate and (D) lactate.

FIG. 2A is a schematic showing the chemical structures of NAD and NADH, and the electron transfer reaction between them.

FIG. 2B is a graph showing the response of NADH at different concentrations in HEPES buffer to PGMs.

FIG. 2C is a schematic showing the use of PGM to detect non-glucose targets based on NADH-dependent enzymatic reactions.

FIGS. 3A-3B are plots showing detection of G6PD activity using PGMs: (A) yeast G6PD in HEPES Buffer; (B) G6PDs from yeast and L. measenteroides in human serum.

FIG. 4 is a dot plot showing the detection of PK activity in human serum using PGMs.

FIG. 5 is a dot plot showing the detection of citrate concentration in HEPES Buffer using PGMs.

FIGS. 6A and 6B are schematic drawings showing how lactate can be detected in a blood sample using a PGM (A) without or (B) with the inclusion of hexokinase.

FIGS. 6C-6E are graphs showing the amplified detection of lactate using PGM by the NADH-based approach. (C) 20 minute reaction time in HEPES buffer; (D) 0.125 mM lactate in HEPES buffer; (E) 20 minutes in human serum.

FIGS. 7A and 7B are plots showing the effect of removing background glucose in (A) HEPES Buffer and (B) human serum.

FIG. 8 is a plot showing the detection of G6PD activity in human serum with or without background glucose removal by hexokinase and ATP.

FIG. 9 is a plot showing the detection of ethanol in human serum using PGM by the NADH-based approach.

FIGS. 10A-10B are graphs showing (A) lactate detection using PGM in HEPES buffer, human serum, and calf blood and (B) validation using UV method in serum and calf blood.

FIG. 10C is a scheme of enzymatic reaction for lactate detection using a UV-vis method.

FIGS. 11A-11B are graphs showing lactate detection in 100% human serum with tunable dynamic range. (A) Reagent A contains 10 μL 50 mM NADH, 10 μL 40 U/mL LOx, 10 μL 200 U/mL LDH, 10 μL 250 mM ATP, 10 μL 250 U/mL hexokinase. (B) Reagent A contains 20 μL 50 mM NADH, 10 μL 40 U/mL LOx, 10 μL 200 U/mL LDH, 10 μL 250 mM ATP, 10 μL 250 U/mL hexokinase.

FIGS. 12A and 12B are graphs showing simultaneous monitoring of (A) glucose and (B) lactate in a blood sample from a subject with diabetes during clinical treatments using PGM and clinical lactate analyzer (YSI 2300 STAT Plus).

FIG. 13 is a graph showing validation of the disclosed PGM-based methods to detect lactate as compared to a clinical lactate analyzer

FIG. 14 is a schematic drawing showing how 3-hydroxybutyrate can be detected in a sample using a PGM.

FIG. 15 is a schematic drawing showing an exemplary disposable lateral flow strip for detection of a target (such as lactate or 3HB) using a commercial glucose meter.

FIGS. 16A-16C show the (A) enzymatic reactions for NADH-PGM based sensor for pyruvate, and pyruvate sensor performance in the (B) presence or (C) and absence of LOx.

FIGS. 17A-17C show the (A) enzymatic reactions for NADH-PGM based sensor for ADP, (B) time-dependent PGM signal decrease in the presence of 1.7 μM ADP, and (C) a calibration curve for ADP.

FIGS. 18A-18B show the (A) enzymatic reactions for NADH-PGM based sensor for ATP, (B) time-dependent PGM signal decrease in the presence of 1.4 μM ATP.

FIG. 19A is a schematic drawing showing enzymatic reactions for NADH-PGM based sensor for creatinine.

FIG. 19B is a graph showing creatinine detection in HEPES buffer with detection limit of 10 μM.

FIG. 20 is a schematic drawing showing an exemplary lateral flow device for the detection of a target enzyme or metabolite in a sample.

FIG. 21 is a schematic drawing showing an exemplary fluidic device for the detection of a target enzyme or metabolite in a sample.

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references.

All publications, patent applications, patents, and other references mentioned herein are incorporated by reference in their entirety for all purposes. All sequences associated with the GenBank Accession numbers mentioned herein are incorporated by reference in their entirety as were present on Nov. 8, 2013. Although exemplary GENBANK numbers are listed herein, the disclosure is not limited to the use of these sequences. Many other enzyme sequences are publicly available, and can thus be readily used in the disclosed methods. In one example, an enzyme having at least 70%, at least 75%, at least 80%, at least 85%, at least 90%, at least 95%, at least 98%, or at least 100% sequence identity to any of the GENBANK numbers are listed herein.

In order to facilitate review of the various embodiments of the disclosure, the following explanations of specific terms are provided:

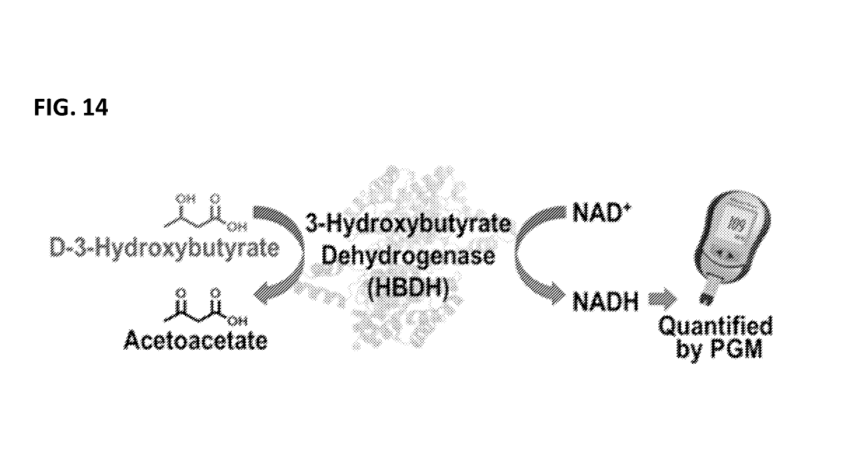

3-hydroxybutyrate dehydrogenase (HBDH): (EC 1.1.1.30) An enzyme that catalyzes the conversion of the chemical reaction shown in FIG. 14. Thus, the substrates for HBDH are 3-hydroxybutanoate (also called 3-β-hydroxybutyrate or β-hydroxybutyrate) and NAD+, and its products are acetoacetate, NADH, and H+. HBDH is involved in the synthesis and degradation of ketone bodies and butanoate metabolism. The presence of elevated levels of 3-β-hydroxybutyrate can indicate the presence of hyperketonemia or ketoacidosis.

Nucleic acid and protein sequences for HBDH (e.g., OMIM 603063) are publicly available. For example, GENBANK® Accession Nos.: BC095414.1 and BC011964.1 (human) and NM_001122683.1 and NM_175177.4 (mouse) disclose exemplary HBDH nucleic acid sequences, and GENBANK® Accession Nos.: Q02338.3 and NP_064524.3 (human) and Q80XN0.2 and NP_001165526.1 (mouse) disclose exemplary HBDH protein sequences, all of which are incorporated by reference as provided by GENBANK® on Nov. 6, 2014. In certain examples, HBDH has at least 80% sequence identity, for example at least 85%, at least 90%, at least 95%, or at least 98% sequence identity to a publicly available HBDH sequence, and is a HBDH which can catalyze the oxidation of 3-hydroxybutanoate to yield acetoacetate, converting NAD+ into NADPH, at the same time.

Coenzyme: An organic, non-protein molecule that binds to a protein (such as G6PD) to form the active protein or enzyme. Coenzymes are required for the biological activity of their corresponding proteins. Coenzymes and enzyme co-factors (inorganic non-protein molecules, such as Zn and Fe), assist in biochemical transformations. Examples of coenzymes include but are not limited to: nicotinamide adenine dinucleotide (NADH), nicotinamide adenine dinucleotide phosphate (NADPH), flavin adenine dinucleotide (FADH₂), and flavin mononucleotide (FMNH₂), as well as derivatives of these that retain their ability to function as a coenzyme, for example by adding one or more additional atoms (such as a carbon, for example a methyl or ethyl group) (other examples can be found in U.S. Pat. No. 8,809,013).

Creatininase: (EC 3.5.2.10) An enzyme that catalyzes the conversion of the first chemical reaction shown in FIG. 22. Thus, the substrates for creatininase are creatinine and H₂O, and its product is creatine. Creatininase is involved in arginine and proline metabolism.

Nucleic acid and protein sequences for creatininase (Gene Ontology No. 0047789) are publicly available. For example, GENBANK® Accession Nos.: AF164677.2 discloses and exemplary creatininase nucleic acid sequence, and GENBANK® Accession Nos.: AGA73231.1 and ETM64754.1 disclose exemplary creatininase protein sequences, all of which are incorporated by reference as provided by GENBANK® on Nov. 6, 2014. In certain examples, creatininase has at least 80% sequence identity, for example at least 85%, at least 90%, at least 95%, or at least 98% sequence identity to a publicly available creatininase sequence, and is a creatininase which can catalyze the hydrolysis of creatinine to yield creatine.

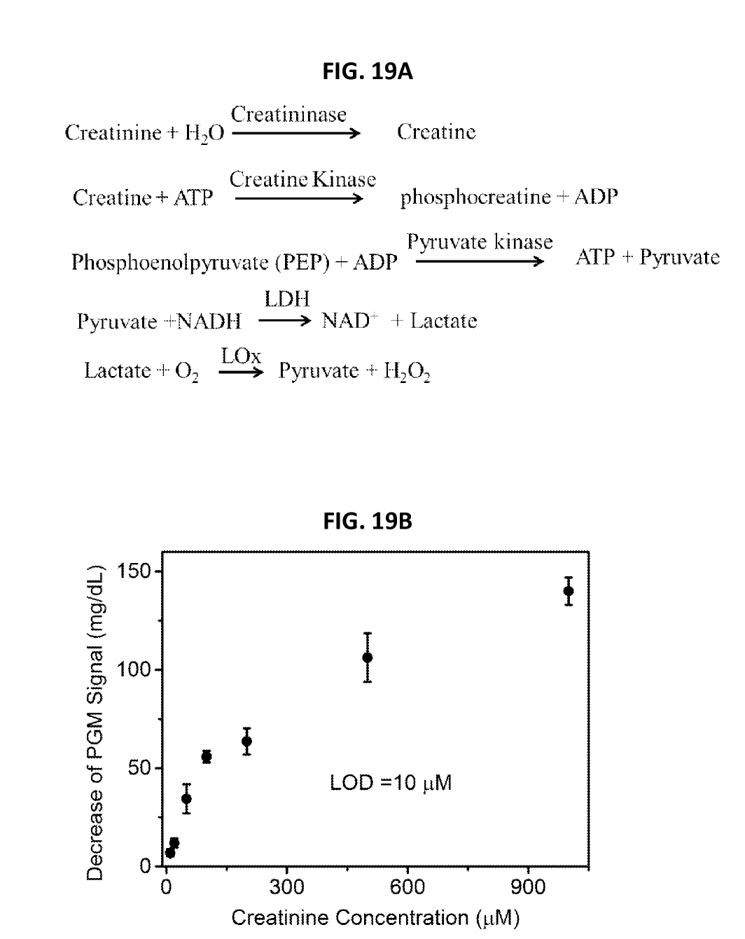

Creatine Kinase (CK): (EC 2.7.3.2) An enzyme that catalyzes the conversion of the chemical reaction shown in the first reaction shown in FIG. 19A. Thus, the substrates for CK are creatine and ATP, and its products are phosphocreatine and ADP. Clinically, CK is assayed in blood tests as a marker of myocardial infarction (heart attack), rhabdomyolysis (severe muscle breakdown), muscular dystrophy, the autoimmune myositides and in acute renal failure.

Nucleic acid and protein sequences for CK, such as cytosolic or muscle CK (e.g., OMIM 123310) are publicly available. For example, GENBANK® Accession Nos.: NM_001824.4 (human) and NM_007710.2 (mouse) disclose exemplary CK nucleic acid sequences, and GENBANK® Accession Nos.: NP_001815.2, AAH07462.1 (human), AAI32427.1 and NP_031736.1 (mouse) disclose exemplary CK protein sequences, all of which are incorporated by reference as provided by GENBANK® on Nov. 6, 2014. In certain examples, CK has at least 80% sequence identity, for example at least 85%, at least 90%, at least 95%, or at least 98% sequence identity to a publicly available CK sequence, and is a CK which can catalyze the production of phosphocreatine and ADP from creatine and ATP.

Detect: To determine if a particular agent is present or absent, such as a target enzyme or target metabolite that is part of an enzymatic reaction that consumes or generates a coenzyme, and in some example further includes quantification of the agent if detected. In some examples a coenzyme is detected, for example with a glucose meter.

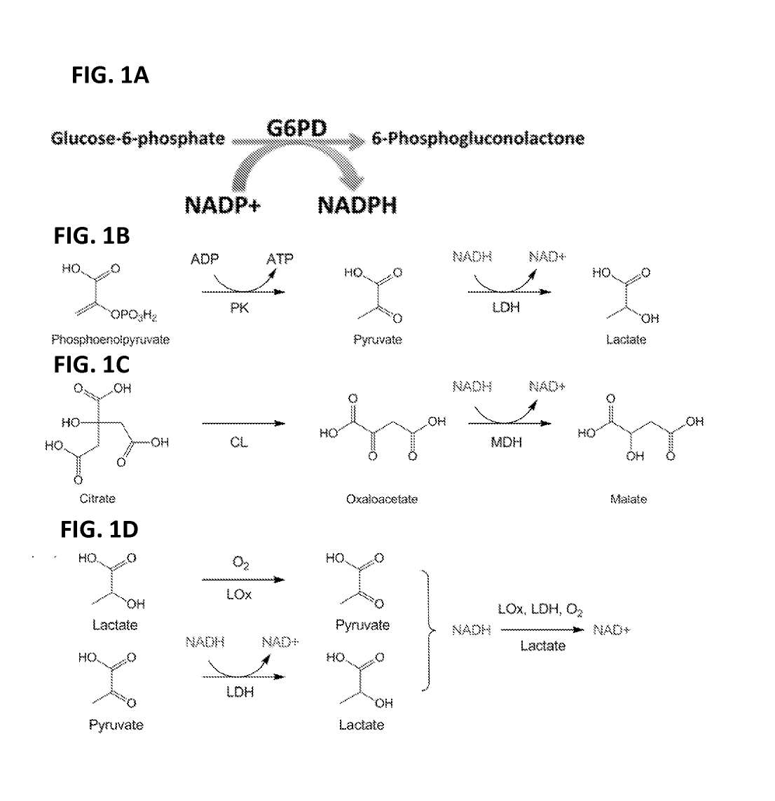

Glucose-6-phosphate dehydrogenase (G6PD): (EC 1.1.1.49) An enzyme that catalyzes the conversion of the chemical reaction shown in FIG. 1A. Thus, G6PD is involved in supplying reducing energy to cells by maintaining the level of NADPH. Deficiencies of G6PD can predispose one to non-immune hemolytic anemia.

Nucleic acid and protein sequences for G6PD (e.g., OMIM 305900) are publicly available. For example, GENBANK® Accession Nos.: AH003054.1 (human) and Z11911.1 (mouse) disclose exemplary G6PD nucleic acid sequences, and GENBANK® Accession Nos.: AAA63175.1 (human) and CAA77967.1 (mouse) disclose exemplary G6PD protein sequences, all of which are incorporated by reference as provided by GENBANK® on Nov. 8, 2013. In certain examples, G6PD has at least 80% sequence identity, for example at least 85%, at least 90%, at least 95%, or at least 98% sequence identity to a publicly available G6PD sequence, and is a G6PD which can catalyze the oxidation of glucose-6-phosphate to yield 6-phosphogluconic acid, converting NAD or NADP into NADH or NADPH, respectively, at the same time.

Glucose Meter: A medical device for determining the approximate concentration of glucose in the blood. Glucose meters include commercially available glucose meters, such as a personal glucose meter (PGM). Such meters typically display the level of glucose in mg/dl or mmol/1. In one example a PGM uses tests strips impregnated with glucose oxidase (GOx; EC 1.1.3.4), such as Bayer Breeze 2®, Medisense Optimum Xeed®, and OneTouch® (such as OneTouch Ultra®, OneTouch Hoizon® or OneTouch Surestep®). In one example a PGM uses tests strips that use glucose dehydrogenase (such as glucose dehydrogenase/pyrroloquinolinequinone GDH/PQQ), such as Precision Xtra®; Ascensia Contour®; Accu-Chek Compact®; Freestyle®, Accu-Chek Avia® and Free Style Lite®. In some examples, a PGMs is one integrated into a mobile phone or other portable platform (such as an iPad®, Surface® or other tablet or a wearable device such as a smart watch or wristband).³⁰ In one example, the PGM is part of (or can be attached to) a cell phone (for example AgaMatrix Inc. (Salem, N.H.) provides a glucose meter that can be attached to a cell phone (such as an iPhone®), and Glooko (Palo Alto, Calif.) provides products that permit one to transfer a reading from a PGM to a cell phone or other device, such as a tablet or a wearable device).

Immobilized: Bound or attached to a surface, such as a solid support. In one embodiment, the solid surface is in the form of a membrane or holding chamber. The surface can include immobilized starting products needed for an enzymatic reaction that utilizes the target enzyme or target metabolite and consumes or generates the coenzyme. Methods of immobilizing agents to solid supports are known in the art. For example, methods of immobilizing peptides on a solid surface can be found in WO 94/29436, and U.S. Pat. No. 5,858,358. In some examples, agents are immobilized to a support by simply applying the agent in solution to the support, and allowing the solution to dry or by lyophoilization, thereby immobilizing the agent to the support.

Lateral flow device: An analytical device in the form of a test strip used in lateral flow chromatography, in which a sample fluid, such as one to be tested for the presence of a target agent, flows (for example by capillary action) through the strip (which is frequently made of bibulous materials such as paper, nitrocellulose, and cellulose). The test sample and any suspended target agent(s) can flow along the strip to a detection zone in which a coenzyme is detected, to indicate a presence, absence and/or quantity of the target agent.

Numerous lateral flow analytical devices are known, and include those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; 6,368,876; 7,799,554; EP 0810436; and WO 92/12428; WO 94/01775; WO 95/16207; and WO 97/06439, each of which is incorporated by reference.

Lateral flow devices can in one example be a one-step lateral flow assay in which a sample fluid is placed in a sample or wicking area on a bibulous strip (though, non bibulous materials can be used, and rendered bibulous by applying a surfactant to the material), and allowed to migrate along the strip until the sample comes into contact with one or more reagents, that lead to the interaction between the target in the sample and the starting products, for the production or consumption of a coenzyme. The coenzyme produced or remaining can be detected with a PGM.

In some examples, the strip includes multiple regions for detecting different test agents in the sample (for example in parallel lines or as other separate portions of the device). The test strips can also incorporate control indicators, which provide a signal that the test has adequately been performed, even if a positive signal indicating the presence (or absence) of a target is not achieved.

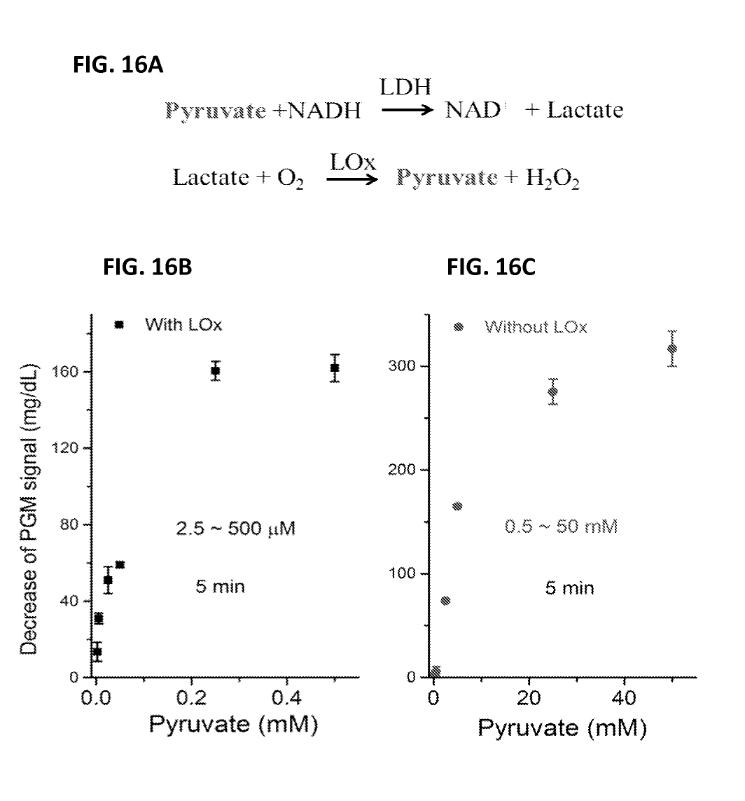

L-Lactate dehydrogenase (LDH): (EC 1.1.2.3) An enzyme that catalyzes the conversion of the chemical reaction shown in the first reaction of FIG. 16A. Specifically, LDH catalyzes the transfer of a hydride group from pyruvate to lactate, and converts NADH to NAD⁺. LDH is released during tissue damage, and thus can be used as a marker of injury and disease.

Nucleic acid and protein sequences for LDH are publicly available. For example, GENBANK® Accession Nos.: CP003592.1 and CP002994.1 disclose exemplary LDH nucleic acid sequences, and GENBANK® Accession Nos.: AFY70884.1 and AFZ53918.1 disclose exemplary LDH protein sequences, all of which are incorporated by reference as provided by GENBANK® on Nov. 6, 2014. In certain examples, LDH has at least 80% sequence identity, for example at least 85%, at least 90%, at least 95%, or at least 98% sequence identity to a publicly available LDH sequence, and is a LDH which can catalyze the transfer of a hydride group from pyruvate to lactate, and convert NADH to NAD+.

Lactate oxidase (LOx): (EC 1.13.12.4) An enzyme that catalyzes the conversion of the chemical reaction shown in the second reaction of FIG. 16A. Specifically, LOx catalyzes the oxidation of lactate, yielding pyruvate and H₂O₂.

Nucleic acid and protein sequences for LOx are publicly available. For example, GENBANK® Accession Nos.: BX950851.1 and CP002279.1 disclose exemplary LOx nucleic acid sequences, and GENBANK® Accession Nos.: WP_024862288.1 and BAA09172.1 disclose exemplary LOx protein sequences, all of which are incorporated by reference as provided by GENBANK® on Nov. 6, 2014. In certain examples, LOx has at least 80% sequence identity, for example at least 85%, at least 90%, at least 95%, or at least 98% sequence identity to a publicly available LOx sequence, and is a LOx which can catalyze the conversion of lactate+O₂ to pyruvate+H₂O₂.

Pyruvate Kinase (PK): (EC 2.7.1.40) An enzyme that catalyzes the conversion of the chemical reaction shown in FIG. 1B. Specifically, PK catalyzes the transfer of a phosphate group from phosphoenolpyruvate (PEP) to ADP, yielding one molecule of pyruvate and one molecule of ATP. Deficiencies of PK can slow down the process of glycolysis, which can be devastating in cells that lack mitochondria, because these cells must use anaerobic glycolysis as their sole source of energy. One example is red blood cells, which in a state of PK deficiency rapidly become deficient in ATP and can undergo hemolysis. Therefore, pyruvate kinase deficiency can cause hemolytic anemia.

Nucleic acid and protein sequences for PK (OMIM 609712) are publicly available. For example, GENBANK® Accession Nos.: M15465.1 (human) and D63764.1 (mouse) disclose exemplary PK nucleic acid sequences, and GENBANK® Accession Nos.: AAA60104.1 (human) and A23642.1 (mouse) disclose exemplary PK protein sequences, all of which are incorporated by reference as provided by GENBANK® on Nov. 8, 2013. In certain examples, PK has at least 80% sequence identity, for example at least 85%, at least 90%, at least 95%, or at least 98% sequence identity to a publicly available PK sequence, and is a PK which can catalyze the transfer of a phosphate group from PEP to ADP, yielding one molecule of pyruvate and one molecule of ATP.

Sensor: A device or part of a device used in the methods provided herein for detecting the presence of a target enzyme or target metabolite, such as one that is part of an enzymatic reaction that consumes or generates a coenzyme. The disclosed sensors can include one or more starting products needed for an enzymatic reaction that utilizes the target enzyme or target metabolite and consumes or generates the coenzyme attached to, or encased in, a solid support (such as a filter, bead or chamber).

Subject: Multi-cellular vertebrate organisms, a category that includes human and non-human mammals, birds, and veterinary subjects (e.g., cows, pigs, dogs and cats).

Target Enzyme or Target Metabolite: An enzyme or metabolite that is part of an enzymatic reaction that consumes or generates a coenzyme, and whose detection is desired. Examples include, but not limited to, G6PD, PK, citrate, ethanol, lactate, 3-β-hydroxybutyrate (3HB), pyruvate, adenosine diphosphate (ADP), adenosine triphosphate (ATP), creatinine, L-lysine, and xanthine. Other examples are provided in Table 1, and one skilled in the art can identify other targets based on the teachings herein. Such targets can be detected indirectly in a sample, by detecting the corresponding production or consumption of a coenzyme using a glucose meter. The detection of such target enzymes or metabolites can be used for applications that include, but are not limited to, disease diagnosis, disease monitoring, drug monitoring (for example levels or amounts of mycophenolate can be determined by monitoring the inhibition of its targeted enzyme, inosine monophosphate dehydrogenase, which utilizes NADH/NAD⁺ as the coenzyme), food safety, and environmental monitoring.

Under Conditions Sufficient For: A phrase that is used to describe any environment that permits the desired activity. In one example, includes incubating a test sample (such as blood or urine) in the presence of one or more starting products needed for an enzymatic reaction that utilizes the target enzyme or target metabolite and consumes or generates the coenzyme under conditions sufficient for the enzymatic reaction to occur, and produce or consume the coenzyme.

### Overview

Personal glucose meters (PGMs) are currently the most successfully commercialized public meters for portable self-diagnosis. PGMs are simple, inexpensive, quantitative, and widely available for worldwide public use. The successful integration of PGMs with mobile phones and tablets allows even wider adoption and better user experience. Currently however, PGMs are only used to help diabetes patients monitor blood glucose. Although other targets can be efficiently detected in research and medical laboratories, they generally require high cost and long time lag for the public to send the samples and wait for the result.

To adapt PGMs to detect other targets, the present disclosure provides methods and sensors that permit detection (and in some examples quantification) of other targets besides glucose. The disclosed methods and sensors enable point-of-care (POC) detection of a wide range of targets, such as disease-related enzymes and non-glucose metabolites. The inventors have determined that coenzymes NADH and NADPH (such as that present in human serum) display a similar response as glucose to PGMs because NADH/NADPH can provide electrons to the electrodes in PGM (while NAD⁺/NADP⁺ do not). In fact, it was observed that NADH generates a higher signal response than glucose at the same concentration because NADH can transfer electrons directly to electrode but glucose has an intermediate step. Since PGM-detectable NADPH/NADH are cofactors in many enzymatic reactions, their production or consumption can be used to measure enzyme activities or substrate concentrations. It is shown herein that NADH or NADPH can be measured as an indication of the presence or absence of a target (for example by measuring increases or decreases in NADH or NADPH).

By using NADH or NADPH as the link, the range of enzymes and metabolites that PGMs can detect is significantly extended. For example, it is shown herein that glucose-6-phosphate dehydrogenase (G6PD), pyruvate kinase (PK), citrate and lactate can be quantified in buffer, calf blood, and/or human serum using PGMs by the disclosed methods, achieving sensitivity well below the clinic cut-off ranges for the diagnosis of G6PD deficiency, PK deficiency, kidney stone risk and lactic acidosis, respectively. It is also shown that pyruvate, adenosine diphosphate (ADP), and adenosine triphosphate (ATP) can be detected and quantified using the disclosed methods. Compared with traditional methods based on UV absorption and fluorescence measurement of NADH (or NADPH), the disclosed sensors and methods are more suitable for POC applications because of the simplicity and portability of PGMs and the absence of separation procedures to remove optically interfering species such as hemoglobin. In addition, the disclosed assays can in some examples be completed within 10 or 20 minutes and have shown high sensitivities well below the cut-off concentrations for diagnosis of various metabolic diseases, indicating their potential in point-of-care (POC) diagnosis of patient samples. Taking advantage of the wide availability of PGMs to the public and their recent integration in mobile phones and tables, as well as the broad range of NAHD-dependent disease-related enzymes and metabolites, the disclosed sensors and methods have numerous useful applications for POC diagnosis of various diseases.

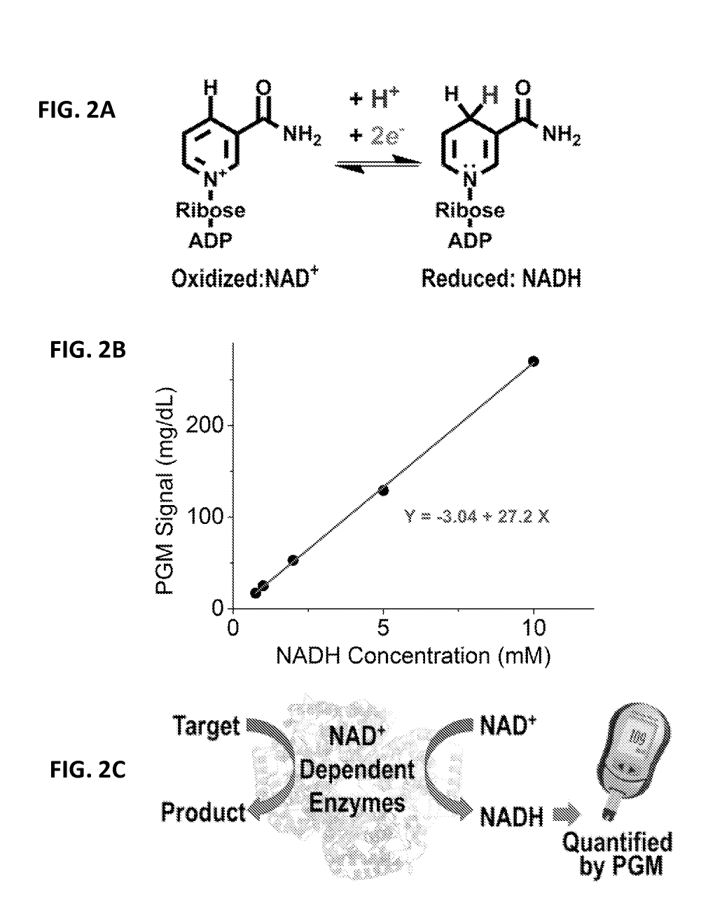

Nicotinamide adenine dinucleotide (NAD) is an enzyme cofactor involved in many enzymatic reactions that are essential for human metabolism. Its conversion between NAD+ and NADH (FIG. 2A) provides or withdraws electrons in a variety of biologically important redox reactions. Several disease-related enzymes and metabolites, including the four targets examined, are capable of facilitating the inter-conversion between NAD+ and NADH (FIGS. 1A-1D).^{11-14,31,32} Although the characteristic UV absorption and fluorescence of NADH have been used as the basis for the traditional enzyme activity and metabolite assays when NADH is formed or deformed in the presence of these targets, few portable devices suitable for point-of-care applications are commercially available. Moreover, the optical signals of NADH in the traditional assays are likely to be interfered by the color of the samples, for example, the hemoglobin in blood. Procedures used to remove the colored species, such as centrifuging or ultra filtering are not suitable for point-of-care applications. Thus, in some examples the disclosed methods do not require centrifuging or ultra filtration steps.

Based on the observations by detecting NADH and NADPH as a means to detect a target in a sample, the present disclosure provides methods of detecting a target enzyme or target metabolite that is part of an enzymatic reaction that consumes or generates an enzyme cofactor or coenzyme (sometimes referred to herein as simply coenzyme), such as an enzymatic reaction involved in metabolism (e.g., in a mammal). Thus, the target enzyme or metabolite is one recognized by enzymatic reactions that convert the concentration of target enzyme or metabolite in the sample into an enzyme cofactor/coenzyme concentration, which is then measured or detected by a PGM. Coenzymes, such as NADH, are non-protein chemical compounds that bind to a protein (such as G6PD) and are required for the biological activity of the protein. These enzyme cofactors and coenzymes assist in biochemical transformations. In addition to NADH and NADPH, other examples of coenzymes or enzyme cofactors that can be detected using the disclosed methods, sensors and devices, include but are not limited to: flavin adenine dinucleotide (FADH₂) (such as its reduced form), flavin mononucleotide (FMNH₂) (such as its reduced form), coenzyme Q, ubisemiquinone (QH radical), ubiquinol (hydroquinone), molybdopterin, and menaquinone, wherein the coenzyme is directly proportional or inversely proportional to an amount of target in the test sample. The disclosed sensors, devices and methods can be designed to detect any target enzyme or target metabolite that is part of an enzymatic reaction that consumes or generates an enzyme cofactor or coenzyme. Exemplary targets are provided herein (e.g., see Table 1); however one skilled in the art will appreciate that other targets can be detected. This disclosure permits the detection of many different targets using a single PGM (for example by using interchangeable lateral flow devices, macro-fluidic devices, micro-fluidic devices, or test strips, each specific for a particular target).

The method includes contacting a test sample with one or more starting products needed for the enzymatic reaction that utilizes the target enzyme or target metabolite and consumes or generates the coenzyme (e.g., one or more enzymes [such as 1, 2, 3, 4, or 5 enzymes], and one or more of ATP, ADP, MgCl₂, NAD+, NADP+ and the like). The enzymatic reaction is allowed to consume or generate the coenzyme, for example by incubating the sample and the starting products under suitable conditions (e.g., temperature, pH). The coenzyme is then detected with a personal glucose meter (PGM). Thus, as the coenzyme is generated, it is detected, while if the coenzyme is consumed, it may not be detected (or its levels may decrease). A determination is made as to whether the target enzyme or metabolite is present in the test sample by correlating the amount of coenzyme detected. The amount of coenzyme detected can be directly or inversely proportional to the amount of target in the sample. The method can determine or measure the amount of target enzyme or target metabolite in the test sample quantitatively or qualitatively. In some examples, blood or fractions thereof (such as serum), or urine are used as the test sample in which the target is detected.

The starting products used will depend on the target to be detected. For example, proteins, cofactors/coenzymes, and energy (e.g., ATP) can be used. For example, if the target enzyme is glucose-6-phosphate dehydrogenase (G6PD), the one or more starting products can include G6P and NAD+ or NADP+; if the target enzyme is pyruvate kinase (PK), the one or more starting products can include phosphoenolpyruvate (PEP), adenosine diphosphate (ADP), NADH and lactate dehydrogenase (LDH); if the target metabolite is citrate, the one or more starting products can include citrate lyase (CL), acetyl coenzyme A, malate dehydrogenase (MDH), NADH and lactate dehydrogenase (LDH); if the target metabolite is lactate, the one or more starting products can include lactate oxidase (LOx), LDH and NADH; if the target metabolite is ethanol, the one or more starting products can include alcohol dehydrogenase (ADH) and NAD⁺; if the target metabolite is lactate, the one or more starting products can include lactate oxidase (LOx), LDH and NADH; if the target metabolite is 3-β-hydroxybutyrate (3HB), the one or more starting products is include 3-hydroxybutyrate dehydrogenase (HBDH) and NAD+; if the target metabolite comprises pyruvate and the one or more starting products can include LDH, LOx, and NADH (in some examples the oxygen needed is supplied simply from the air); or if the target metabolite is creatinine and the one or more starting products can include creatininase, ATP, creatine kinase, PK, PEP, LDH, LOx, and NADH (in some examples the oxygen needed is supplied simply from the air). The starting products, such as enzyme(s), substrates(s), NADH or NAD⁺, can be added to the sample tested or be present in a sensor to which the sample (or portion thereof) is applied.

In one example, the method further includes steps to remove glucose from a sample, such as a blood or urine sample. For example, the method can include contacting the sample with hexokinase, MgCl₂, and ATP (e.g., which can be present in or on a sensor, such as a lateral flow strip or fluidic device, or which can be added to the sample prior to contact with such a sensor) under conditions sufficient to remove glucose in the test sample. In some examples, such methods reduce the amount of detectable glucose in the sample by at least 50%, at least 75%, at least 90%, at least 95%, at least 98%, at least 99%, or all of the detectable glucose is removed.

In some examples, the disclosed methods and sensors can be used without the need for laboratory-based instruments or complicated sample pre-treatment. For example, in some examples, the test sample is a blood sample or fraction thereof, and hemoglobin in the sample is not removed prior to analysis of the sample using the disclosed methods.

In some examples, the detected enzyme coenzyme is compared to a control value, such as a reference value or range of values. In one example, the control value is a reference value or range of values for the target enzyme or metabolite expected in a normal subject (e.g., one without a G6PD deficiency). In one example, the control value is a value (or range of values, e.g., from a population) for the target enzyme or metabolite determined from a control sample, such as one obtained from a normal subject (e.g., one without a G6PD deficiency) or population of subjects. Thus, the method can further include detecting the target enzyme or target metabolite in a normal control sample, and comparing the target enzyme or target metabolite detected in the test sample to that detected in the normal control sample. Such steps allow for a determination as to whether the target enzyme or metabolite in the test sample is increased or decreased relative to a normal subject. In one example, the control value is value for the target enzyme or metabolite determined from a control sample, such as one obtained from a sample with a known amount of the target enzyme or metabolite (e.g., positive control) or known to not have the target enzyme or metabolite (e.g., negative control).

In some examples, the test sample is obtained from a subject, and the method includes determining whether the subject has a disease based on the amount of target enzyme or target metabolite detected. For example, using the method to detect G6PD can be used to diagnose G6PD deficiency in a subject, wherein the subject is determined to have G6PD deficiency when an increase in NADH or NADPH relative to a normal control is detected. Using the method to detect PK can be used to diagnose PK deficiency in a subject, wherein the subject is determined to have PK deficiency when a decrease in NADH or NADPH relative to a normal control is detected. Using the method to detect citrate can be used to diagnose prostate cancer or kidney stones or risk of developing kidney stones, wherein the subject is determined to have prostate cancer, kidney stones or an increase risk of developing kidney stones, when a decrease in NADH or NADPH relative to a normal control is detected. Using the method to detect lactate can be used to diagnose lactic acidosis, wherein the subject is determined to have lactic acidosis when a decrease in NADH or NADPH relative to a normal control is detected. Using the method to detect ethanol can be used to diagnose ethanol poisoning, wherein the subject is determined to have ethanol poisoning when an increase in NADH or NADPH relative to a normal control is detected. Using the method to detect ethanol can be used to determine a subject's blood alcohol level (for example for a DUI or DWI evaluation), wherein the subject's alcohol level is proportional to the amount of NADH or NADPH detected. Using the method to detect 3-β-hydroxybutyrate (3HB) can be used to determine whether a subject has ketoacidosis, wherein the subject's 3-β-hydroxybutyrate (3HB) level is proportional to the amount of NADH or NADPH detected. Using the method to detect pyruvate can be used to determine whether a subject has pyruvate kinase deficiency, wherein the subject is determined to have pyruvate kinase deficiency when a decrease in NADH or NADPH relative to a normal control is detected. Using the method to detect creatinine can be used to determine whether a subject has renal disease, wherein the subject is determined to have renal disease when a decrease in NADH or NADPH relative to a normal control is detected.

The present disclosure also provides sensors that can be used in the disclosed methods. Such a sensor can include a solid support to which is attached or which includes one or more starting products needed for an enzymatic reaction that utilizes the target enzyme or target metabolite and consumes or generates the coenzyme. Such starting products can be incorporated into a solution, such as a water-based solution, and dried or lyophilized on the solid support. Thus, in some examples the starting products are soluble. In some examples, the sensor further includes a second solid support that includes reagents to substantially remove glucose from the sample, such as hexokinase and ATP (and optionally MgCl₂). Such reagents can be incorporated into a solution, such as a water-based solution, and dried or lyophilized on the solid support. Thus, in some examples the reagents are soluble. In some examples, the sensor further includes a sample pad and an absorption pad. In some examples, the sensor further includes one or more mixing chambers, an entry port, and an exit port. In some examples, the solid support includes a membrane or a holding chamber. In some examples, the solid support can be polymer beads, such as agarose, sepharose beads, magnetic beads, or the chamber walls of the fluidic device.

The disclosure also provides, lateral flow devices and fluidic devices (e.g., macro- and micro-fluidic devices) that can be used with the disclosed methods. In some examples, such devices include a sensor provided herein. In one example, a fluidic device includes a sample entry port, a holding chamber containing the one or more starting products, optionally one or more mixing chambers, and an exit port. In one example, a a fluidic device includes a sample entry port, a one or more mixing chambers, one or more filters and an exit port. For example, such a a fluidic device can include a first mixing chamber that includes buffer reagents (e.g., HEPES, MgCl₂), pretreatment reagents (e.g., anticoagulation agents, detergent(s), hexokinase), or both, and a second mixing chamber that includes starting products needed for an enzymatic reaction that utilizes the target enzyme or target metabolite and consumes or generates the coenzyme (e.g., coenzymes or cofactors such as ATP, ADP, LDH, NADH, and NADPH). In some examples there is a filter in between the first and second mixing chambers, for example to remove cellular debris and large proteins.

Also provided are methods for using such lateral flow devices, and fluidic devices to detect a target enzyme or target metabolite. For example, the method can include contacting one or more sensors or lateral flow devices with a test sample under conditions sufficient to allow target enzyme or target metabolite in the test sample to interact with the one or more starting products, under conditions wherein the coenzyme will be generated or consumed, detecting the enzyme cofactor or coenzyme with a PGM, and correlating the amount of target agent present in the sample to the amount of enzyme cofactor or coenzyme detected. In another example, the method can include introducing a test sample into the sample entry port of a fluidic device, allowing a target in the test sample to interact with a holding or mixing chamber of the device containing the one or more starting products, allowing the cofactor or coenzyme to be generated or consumed in one of the one or more mixing chambers of the device, detecting the enzyme cofactor or coenzyme with a PGM, and correlating the amount of target agent present in the sample to the amount of enzyme cofactor or coenzyme detected.

Also provided are kits that include one or more of the sensors lateral flow devices, or fluidic devices disclosed herein. Such kits in some examples further include one or more of a buffer, a chart for correlating detected enzyme cofactor or coenzyme level and amount of target enzyme or target metabolite present. In some examples, the kit includes a PGM.

### Methods of Detecting Target Enzymes or Metabolites

Methods are provided for detecting a target enzyme or target metabolite that is part of an enzymatic reaction that consumes or generates an enzymatic cofactor or coenzyme (referred to herein as a “coenzyme”), such as an enzymatic reaction involved in human metabolism. Thus, the target enzyme or metabolite is a component of an enzymatic reaction, wherein the reaction converts the concentration of target enzyme or metabolite in the sample into a coenzyme concentration, which is then measured or detected by a PGM. In some examples, for example when a sensor or lateral flow device is used, the method can include contacting the lateral flow device with a sample under conditions sufficient to allow the target agent in the sample to flow through the lateral flow device and interact with various reagents present on the lateral flow device, including starting products needed for the enzymatic reaction that utilizes the target. Consumption or reduction of the coenzyme is detected, for example with a PGM. In some examples, for example when macro- or micro-fluidic device is used, the method can include introducing the test sample into the device under conditions sufficient to allow the target agent in the sample to flow through the device and interact with various reagents present in or introduced into the device (such as those present in one or more mixing chambers), including starting products needed for the enzymatic reaction that utilizes the target. If present, the coenzyme exits the device through a port, and can be detected for example with a PGM.

In some examples, the method includes contacting a test sample (such as a blood sample or urine sample or other type of sample, such as an environmental or food sample) with one or more starting products, wherein the starting products are components of the enzymatic reaction that utilizes the target enzyme or metabolite. In some examples, the method uses at least two starting products (such as three or more, or four or more, such as 1, 2, 3, 4, 5, 6, 7, 8, 9 or 10 different starting products, such as (a) an enzyme or substrate (that which is acted upon by an enzyme) and (b) NAD⁺ or NADH). For example, if the target is a metabolite, the starting products can include at least one enzyme (such as 1, 2, 3, 4, 5, 6, 7, 8, 9 or 10 different enzymes) and NAD⁺ or NADH. If the target is an enzyme, the starting products can include at least one substrate (such as 1, 2, 3, 4, 5, 6, 7, 8, 9 or 10 different substrates) and NAD⁺ or NADH. The test sample and one or more starting products are incubated under conditions that allow the enzymatic reaction that utilizes the target enzyme or metabolite to consume or generate a coenzyme. The method then includes detecting the coenzyme with a PGM, and correlating the amount of coenzyme detected to an amount of target present in the test sample. Detecting the coenzyme can be qualitative or quantitative.

In particular examples, the sample is obtained from a subject, such as a human subject. In such examples, the method can further include obtaining the sample from the subject. In some examples, the method includes selecting a subject having or suspected of having a particular disease associated with the target enzyme or metabolite. In some examples, the method further includes determining that the subject has a particular disease depending on whether the target enzyme or metabolite is detected or not (or is increased or decreased relative to an appropriate normal control or reference value, such as a sample from a subject that does not have the disease screened for or a reference value indicting absence of the test agent). In some examples, the method includes treating the subject for the disease diagnosed using the disclosed methods.

In some examples, the method includes detecting or determining a level or amount of, for example in a subject who received the drug. Mycophenolate can be detected by monitoring the inhibition of its targeted enzyme, inosine monophosphate dehydrogenase, which utilizes NADH/NAD⁺ as the coenzyme.

In particular examples, the sample is obtained from an environmental or food source. In such examples, the method can further include obtaining the sample from the environmental or food source. In some examples, the method includes selecting an environmental or food source containing or suspected of containing a particular pathogen associated with the target enzyme or metabolite.

In some examples, detection of the coenzyme indicates the presence of the target in the sample, and an absence of detected coenzyme indicates the absence of the target in the sample (directly proportional). In other examples, detection of coenzyme indicates the absence of the target in the sample, and an absence of detected coenzyme indicates the presence of the target in the sample (inversely proportional). In some examples, the coenzyme detected is compared to a control, such as a reference value(s) indicating the presence or absence of the target or samples known to have or not have the target (such as a reference sample or plurality of samples containing a known amount of target or a sample known not to contain the target).

In some examples, the disclosed methods include neutralizing the resulting reaction, for example from a basic pH to a neutral pH (for example to a pH of about 6.5 to 7.5, such as pH 6.8 to 7.2), after the coenzyme is produced or consumed, but before it is detected with a glucose meter. For example the reaction can be incubated or contacted with a buffer containing phosphate buffers (e.g., sodium, potassium or ammonium salts, such as NaH₂PO₄ and the like), and other buffers such as Tris-HCl, HEPES, MES, NaHCO₃—Na₂CO₃, and the like.

In some examples, the sample is incubated with reagents to remove glucose from the test sample, such as incubation with hexokinase, MgCl₂, and ATP. Hexokinase is an enzyme capable of converting glucose into glucose-6-phosphate in the presence of ATP. Glucose-6-phosphate is not detectable in glucose meters and ATP does not interfere with glucose detection. Thus, in some examples the sensors provided herein include hexokinase (and in some examples also MgCl₂ and ATP), for example on a solid support (such as a reagent pad) or in a mixing chamber.

In some examples, the sample is incubated with agents to increase the amplification of the coenzyme, such as NADH. For example, to detect lactate, the step of NADH conversion to NAD+ can be amplified by using lactate oxidase (LOx) to convert lactate to pyruvate, and the pyruvate is further changed back to lactate by LDH to consume one NADH. In this case, one lactate can be used many times in the reaction to induce the consumption of multiple NADHs.

In particular examples, the PGM used is one that uses a test strip that includes glucose oxidase (GOx). Examples of PGMs that use GOx include but are not limited to: Bayer Breeze 2®, Medisense Optimum Xeed®, and OneTouch® (such as OneTouch Ultra®, OneTouch Horizon® or OneTouch Surestep®). In particular examples, the PGM used is one that does not use a test strip that includes glucose dehydrogenase (such as glucose dehydrogenase/pyrroloquinolinequinone GDH/PQQ). Thus, in some examples, the test strip used has glucose oxidase or glucose dehydrogenase. However, in some examples, the test strip used does not include these enzymes, as the bare test strips (e.g., electrodes) can detect redox-active cofactors (e.g., coenzymes such as NADH or NADPH).

Detecting Target Agents that Generate or Consume NADH or NADPH

The targets that can be detected with the disclosed methods, devices, and sensors include those that are part of an enzymatic reaction that consumes or generates NADH or NADPH, as both can be detected with a PGM. Thus, the presence of targets that are part of an enzymatic reaction that consume NADH or NADPH can be detected by detecting a decrease in NADH or NADPH, while targets that are part of an enzymatic reaction that generate NADH or NADPH can be detected by detecting a change (e.g., increase) in NADH or NADPH.

In one example, the target is an enzyme that consumes or generates NADH or NADPH as part of an enzymatic reaction (such as where NADH/NAD⁺ or NADPH/NADP⁺ conversion is involved). Examples of such enzymes, include, but are not limited to: (1) oxidoreductases (dehydrogenases), such as glucose-6-phosphate oxidase; lactate dehydrogenase; amino acid dehydrogenase (such as glutamate dehydrogenase); malate dehydrogenase; alcohol dehydrogenase; glyceraldehyde-3-phosphate dehydrogenase; alpha-ketoglutarate dehydrogenase; and the like, and (2) enzymes related to the substrates of oxidoreductases, such as pyruvate kinase; galactose-1-phosphate uridylyltransferase; phenylalanine hydroxylase; phosphoglucomutase; hexokinase; glucose isomerase; glucose-6-phosphate isomerase; urease; and the like. Other examples are listed in Table 1.

In one example, the target is a metabolite that serves as a substrate of an enzymatic reaction which consumes or generates NADH or NADPH (such as where NADH/NAD⁺ or NADPH/NADP⁺ conversion is involved). Examples of such metabolites, include, but are not limited to: pyruvate; ammonia; bilirubin; creatinine; cholesterol; triglycerides; urea; phenylalanine; galactose; and the like. Other examples are listed in Table 1.

As described above, selecting appropriate starting product(s) permits detection of the target enzyme or metabolite, and allows one to develop a sensor that can be used to detect a particular target enzyme or metabolite. If the target enzyme consumes or generates NADH or NADPH directly from a starting product (such as the enzyme G6PD, see FIG. 1A), additional enzymes or materials may not be required. However, if the target enzyme does not consume or generate NADH or NADPH directly (such as the enzyme PK, see FIG. 1B), but instead produces a product in the pathway that can be acted upon by a second enzyme that can consume or generate NADH or NADPH, the second enzyme (or more enzymes as needed) can be supplied (for example with the starting products). If the target metabolite serves as a substrate of an enzymatic reaction that consumes or generates NADH or NADPH directly from a starting product additional enzymes may not be required. However, if the target metabolite serves as a substrate of an enzymatic reaction that does not consume or generate NADH or NADPH directly (such as citrate, see FIG. 1C), but instead produces a product in the pathway that can be acted upon by an enzyme that can consume or generate NADH or NADPH, the second enzyme (or more enzymes as needed) can be supplied (for example with the starting products).

Examples of starting products that can be added to a test sample to detect a particular target are shown in Table 1. Such starting products can be added to the sample by way of a sensor, on or in which the starting products are present (e.g., by contacting the sample with the sensor or introducing the sample into the sensor). In some examples, MgCl₂ is also present in the testing reaction (e.g., can be a further starting product). Diseases that correspond to the enzyme or metabolite are also shown. For many of these enzymes or metabolites, when the level deviates from the normal level, this indicates a problem or disease.

TABLE 1
Exemplary target enzymes and metabolites and diseases that can be detected with the disclosed
methods. Agents that are added to/contacted with the sample are also provided.

| | Additional Reagents Added to | |
|---|---|---|
| Enzyme | Sample (Starting Products) | Disorder |
| glucose-6-phosphate | Glucose-6-phosphate, NADH | G6PD deficiency |
| dehydrogenase | | indicated if a decrease in |
| | | NADH or NADPH |
| | | detected |
| lactate dehydrogenase | Pyruvate, NADH | diagnosis of |
| | | malignancies if |
| | | increased NADH is |
| | | detected (hence |
| | | increased LDH activity) |
| amino acid dehydrogenase | Amino acid, coenzyme Q, alpha- | Glutaryl-CoA |
| | ketoglutamate, NADPH, glutamate | dehydrogenase |
| | dehydrogenase | deficiency (GDD) |
| | | indicated if increased |
| | | NADPH detected |
| malate dehydrogenase | Oxaloacetate, NADH | Liver disease indicated |
| | | if increased NADH |
| | | detected |
| alcohol dehydrogenase | Alcohol, NAD+ | Alcohol dehydrogenase |
| | | deficiency indicated if a |
| | | decrease in NADH |
| | | detected |
| gyceraldehyde-3-phosphate | G3P, NAD+ | neoplasms, adult T-cell |
| dehydrogenase | | leukemias and acute |
| | | leukemias. |
| alpha-ketoglutarate dehydrogenase | Alpha-ketoglutarate, Coenzyme-A, | Neurodegenerative |
| | NAD⁺ | disorders indicated if |
| | | increased NADH |
| | | detected |
| pyruvate kinase | phosphoenolpyruvate (PEP), | PK deficiency indicated |
| | adenosine diphosphate (ADP), | if an increase in NADH |
| | NADH and lactate dehydrogenase | or NADPH detected |
| | (LDH) | |
| phenylalanine hydroxylase (PAH) | Phenylalanine, 6- | Phenylketonuria (PKU) |
| | methyltetrahydropterine | |
| Hexokinase (coverts glucose to | Glucose, ATP, G6P, and NAD+ or | Hexokinase deficiency |
| G6P) | NADP⁺ | indicated if an increase |
| | | in NADH or NADPH is |
| | | detected |
| glucose isomerase | glucose | |
| urease | Urea, alpha-ketoglutamate, | Helicobacter pylori |
| | NADH, glutamate dehydrogenase, | infection indicated if |
| | ADP | increased NADH |
| | | detected |
| Alanine aminotransferase (aka | Lactate dehydrogenase, NADH or | Hepatocellular injury |
| alanine transaminase EC 2.6.1.2) | NAD⁺ | |
| Xanthine oxidase (EC 1.17.3.2) | Xanthine, NADH, NADH | Consumption rate of |
| | peroxidase | NADH indicates the |
| | | activity of xanthine |
| | | oxidase. Lower rate |
| | | indicates the xanthine |
| | | oxidase deficiency, |
| | | indicating high risk of |
| | | xanthinuria |
| Metabolite | | |
| pyruvate | For pyruvate detection, NADH, | Glycolysis |
| | LDH, and LOx | Pyruvate kinase |
| | For detection of pyruvate kinase, | deficiency (PKD) |
| | phosphoenolpyruvate (PEP), | |
| | adenosine diphosphate (ADP), | |
| | NADH and lactate dehydrogenase | |
| | (LDH) | |
| ammonia | Alpha-ketogluraric acid, NADPH, | Liver diseases including |
| | L-glutamate dehydrogenase | cirrhosis and hepatitis |
| bilirubin | Anti-bilirubin monoclonal | Liver diseases |
| | antibody | |
| Creatinine | creatininase, ATP, creatine kinase, | Reduction of NADH |
| | PK, PEP, LDH, LOx, and NADH | indicates the presence of |
| | | creatinine, and thus can |
| | | be used to renal |
| | | function, monitor the |
| | | course of renal disease, |
| | | and adjust renal function |
| | | dependent drug dosages; |
| | | Kidney diseases |
| cholesterol | cholesterol esterase, cholesterol | Decrease of NADH |
| | | indicates the elevation |
| | | of cholesterol, |
| | | indicating risks for |
| | | cardiovascular diseases |
| triglycerides | Lipoprotein Lipase, glycerol | Decrease of NADH |
| | kinase, ATP, PEP, Pyruvate | indicates the elevation |
| | kinase, LDH, NADH | of triglycerides, |
| | | indicating risks for |
| | | cardiovascular disease |
| urea | Urease, alpha-ketoglutamate, | Kidney diseases |
| | NADH, glutamate dehydrogenase, | |
| | ADP | |
| phenylalanine | Phenylalanine hydrolase, 6- | phenylketonuria (PKU) |
| | methyltetrahydropterin | |
| galactose | NAD⁺, galactose dehydrogenase | carcinoma and |
| | | precancerous lesions |
| ethanol | Alcohol dehydrogenase, NAD⁺ | Ethanol poisoning |
| | | indicated if an increase |
| | | in NADH or NADPH is |
| | | detected. |
| Lactate | lactate oxidase (LOₓ), lactate | Decrease of NADH |
| | dehydrogenase (LDH), NADH, | indicates the presence of |
| | ATP | lactate, and can be used |
| | | to diagnose lactic |
| | | acidosis and to |
| | | determine the lactate |
| | | threshold |
| 3-β-hydroxybutyrate (3HB) | 3-hydroxybutyrate dehydrogenase | The presence of NADH |
| | (HBDH), NAD+ | indicates the presence of |
| | | 3HB, and thus increases |
| | | in NADH can be used to |
| | | diagnose or monitor for |
| | | ketoacidosis |
| ADP | Phosphoenolpyruvate, pyruvate | Consumption of NADH |
| | kinase, LDH, NADH, LOx | indicates the presence of |
| | | ADP, which can be used |
| | | to monitor ATPases that |
| | | produce ADP and Pi and |
| | | kinases that produce |
| | | ADP and a |
| | | phosphorylated product |
| ATP | Creatine, creatine kinase, PEP, | Consumption of NADH |
| | pyruvate kinase, LDH, NADH | indicates the presence of |
| | | ATP, which can be used |
| | | to monitor cell injury or |
| | | disease, such as |
| | | angiocardiopathy. |
| L-lysine | Lysine dehydrogenase (EC | Production of NADH |
| | 1.4.1.15), NAD⁺ | indicates the presence of |
| | | L-lysine, and thus can |
| | | be used to detect lysine |
| | | deficiency. |

Thus, markers such as lactate, 3HB, and pyruvate can be used as indicators of diabetes, or determined in a sample from a subject having or suspected of having diabetes (such as type I or type II diabetes), or pre-diabetes. For example, measuring lactate (decreases in NADH) can be used as a marker of lactic acidosis, a side effect of biguanide drugs for diabetes. Measuring 3HB (increases in NADH) can be used as a marker of ketoacidosis. In addition, markers of disease can also be detected, such as creatinine (e.g., to detect kidney disease), alanine aminotransferase (e.g., to detect liver disease), and G6PD (e.g., to detect G6PD deficiency). In addition, small organic molecules can also be detected, such as ADP, ATP, and L-lysine.

Detecting Target Agents that Generate or Consume Other Cofactors

As shown above in Table 1, other targets that can be detected with the disclosed methods, devices, and sensors include those that are part of an enzymatic reaction that consumes or generates other coenzymes besides NADH or NADPH. Thus, the presence of targets shown in Table 1 that are part of an enzymatic reaction that consume the coenzyme can be detected by detecting a decrease in the coenzyme, while targets that are part of an enzymatic reaction that generate the coenzyme can be detected by detecting an increase in the coenzyme. On skilled in the art will appreciate that other coenzymes are known in the art, as are their corresponding enzymatic reactions. Thus, the disclosure is not limited to the detection of particular coenzymes. One skilled in the art can readily apply the principles provided herein to other coenzymes in addition to NADH and NADPH.

Exemplary Disorders that can be Diagnosed

The ability to detect a target enzyme or metabolite permits diagnosis of disorders associated with such targets. In some examples the methods include selecting a subject having or suspected of having a particular disease, such as those disorders described below and throughout the application. In some examples, the sample to be analyzed using the disclosed methods, devices, and sensors, is obtained from a subject having or suspected of having particular disease, such as those disorders described below and throughout the application.

Glucose-6-Phosphate Dehydrogenase (G6PD) Deficiency

The ability to detect NADH/NADPH using the disclosed methods, sensors, and devices permits detection of G6PD and diagnosis of G6PD deficiency. For example, a G6PD deficiency is indicated as a decrease in NADH (or NADPH) as compared to a control, such as a reference value of G6PD expected in a healthy subject without G6PD deficiency. In some examples, normal values of G6PD for an adult are about 5-14 unit/g hemoglobin (e.g., see Nicoll et al. (Eds): Pocket Guide to Diagnostic Tests, 3rd. McGraw-Hill, New York, N.Y., United States, 2001).

In one example, detection of significantly decreased levels of G6PD or decreased levels of NADH (for example relative to a normal control), or no G6PD or NADH (or NADPH), indicates that the patient from which the sample was obtained has G6PD deficiency. For example, if NADH (or NADPH) is decreased by at least 50%, at least 60%, at least 75%, or at least 90%, relative to NADH (or NADPH) detected in a sample from a corresponding normal sample (e.g., non-G6PD deficiency sample), this would lead to a diagnosis of G6PD deficiency in the test patient. For example, if G6PD values are reduced by at least 50%, at least 60%, at least 75%, at least 80%, at least 80%, or at least 90%, relative to G6PD detected in a sample from a normal individual (such as one without G6PD deficiency), this would lead to a diagnosis of G6PD deficiency in the test patient.

G6PD deficiency is the most common human enzyme defect. Subjects with the disease may exhibit nonimmune hemolytic anemia in response to a number of causes, most commonly infection or exposure to certain medications or fava beans. Due to the X-linked pattern of inheritance, most symptomatic patients are male.

Abnormal red blood cell breakdown (hemolysis) in G6PD deficiency can manifest in a number of ways, including prolonged neonatal jaundice, possibly leading to kernicterus; hemolytic crises in response to illness (e.g., infections), drugs (e.g., primaquine, pamaquine, and chloroquine), foods (e.g., broad beans), chemicals or diabetic ketoacidosis; and acute renal failure.

In most case, prevention is used to treat G6PD deficiency (e.g., avoidance of the drugs and foods that cause hemolysis). In some examples, vaccination against some common pathogens (e.g., hepatitis A and hepatitis B) is used to prevent infection-induced attacks. In some examples, blood transfusions are administered, or dialysis can be administered in acute renal failure. Some patients have their spleen removed. Folic acid can be administered. Thus, in some examples, the methods provided herein can further include such prevention and/or treatment.

Pyruvate Kinase (PK) Deficiency

The ability to detect NADH/NADPH using the disclosed methods, sensors, and devices permits detection of G6PD and diagnosis of G6PD deficiency. For example, a PK deficiency is indicated as an increase in NADH (or NADPH) as compared to a control, such as a reference value of PK expected in a healthy subject without PK deficiency. In some examples, a normal PK value is 179±16 units per 100 mL of red blood cells.

Thus, in one example, detection of significantly increased levels of NADH (or NADPH), or decreased levels of PK, for example relative to a normal control indicates that the patient from which the sample was obtained has a PK deficiency. For example, if NADH (or NADPH) is increased by at least 20%, at least 50%, at least 100%, or at least 200%, relative to NADH (or NADPH) detected in a sample from a corresponding normal sample (e.g., non-PK deficiency sample), this would lead to a diagnosis of PK deficiency in the test patient. For example, if PK values are reduced by at least 50%, at least 60%, at least 75%, at least 80%, at least 80%, or at least 95%, relative to PK detected in a sample from a normal individual (such as one without PK deficiency), this would lead to a diagnosis of PK deficiency in the test patient.

Pyruvate kinase deficiency is the second most common cause of enzyme-deficient hemolytic anemia, following G6PD deficiency. RBC lysis can lead to hemolytic anemia and may cause jaundice from increased bilirubin.

Most affected individuals do not require treatment. Individuals may require blood transfusions or splenectomy. Treatment is usually effective in reducing the severity of the symptoms. Thus, in some examples, the methods provided herein can further include such treatment.

Disorders Associated with Citrate Deficiency

The ability to detect NADH/NADPH using the disclosed methods, sensors, and devices permits detection of citrate. Deficiency of citrate in urine indicates a disease, and thus permits diagnosis of several disorders, such as prostate cancer and kidney stones. For example, levels of citrate can be determined by monitoring NADH (or NADPH) concentration as compared to a control, such as a reference value of citrate expected in a healthy subject. Normal values for citrate in the urine are age-dependent, and range from 150 mg to 1200 mg/24 hr secretion in urine, such as 640 mg/day or 3.39 mmol/d (<150 mg/24 hr).

Thus, in one example, detection of significantly reduced levels of citrate or NADH (or NADPH) or no citrate or NADH (or NADPH) (for example relative to a normal control) indicates that the patient from which the sample was obtained has an increase risk for developing kidney stones, has kidney stones, or has prostate cancer. For example, if citrate or NADH (or NADPH) values are reduced by at least 50%, at least 60%, at least 75%, at least 80%, at least 80%, or at least 95%, relative to citrate or NADH (or NADPH) detected in a sample from a normal individual (such as one without citrate deficiency), this would lead to a diagnosis of increase risk for developing kidney stones, has kidney stones, or has prostate cancer in the test patient.

For example, low citrate in human urine (such as values of less than 2 mmol/day, such as less than 1.7, mmol/d, less than 1.5, mmol/d or less than 1.2 mmol/d) is an indication of high risk in kidney stone formation. Thus, detection of decreased NADH or NADPH levels in urine using the methods provided herein can indicate an increased risk of developing kidney stones.

For example, low citrate in human urine (such as values of less than 2 mmol/day, such as less than 1.7, mmol/d, less than 1.5, mmol/d or less than 1.2 mmol/d) or prostatic fluid (normal is about ˜50-200 mM citrate, cancer level is about 2-20 mM, with levels in seminal fluid about 50% of prostatic level) is an indication of prostate cancer. Thus, detection of decreased NADH or NADPH levels in urine or prostatic fluid using the methods provided herein can indicate can indicate the subject has prostate cancer. Thus, in some examples, such a method is used to monitor a patient's prostate cancer before, during and/or following treatment.

Disorders Associated with Increased Ethanol

The ability to detect NADH or NADPH using the disclosed methods, sensors, and devices permits detection of ethanol, and thus permits diagnosis of several disorders, such as ethanol poisoning and toxicity testing. For example, levels of ethanol can be determined by monitoring NADH (or NADPH) concentration as compared to a control, such as a reference value of ethanol expected in a healthy subject (e.g., >0.10% by volume in blood, such as 0%). Such methods can be used for DUI or DWI testing to determine a person's blood-alcohol level.

Thus, in one example, detection of significantly increased levels of ethanol or NADH (or NADPH) (for example relative to a normal control) indicates that the patient from which the sample was obtained has ethanol poisoning. For example, if ethanol or NADH (or NADPH) values are increased by at least 5%, at least 10%, at least 50%, at least 100%, or at least 200%, relative to ethanol or NADH (or NADPH) detected in a sample from a normal individual (such as one without ethanol poisoning), this would lead to a diagnosis of ethanol poisoning in the test patient.

For example, increased ethanol in human blood or fractions thereof (such as values of at least 0.05%, by volume in blood, such as 0.06%, 0.07%, 0.08%, 0.09%, 0.1%, 0.2% 0.3%, 0.4%, or 0.5% by volume in blood, such as 1-10 mM or 5-10 mM), is an indication of ethanol poisoning. Thus, detection NADH levels of at least 6 mM or at least 10 mM in the blood can indicate ethanol poisoning.

In some examples, the methods permit detection of a person's blood alcohol level, such as levels of at least 0.01%, by volume in blood, such as at least 0.02%, at least 0.03%, at least 0.04%, at least 0.05%, at least 0.06%, at least 0.07%, at least 0.08%, at least 0.09%, at least 0.1%, at least 0.15%, at least 0.2%, at least 0.3%, at least 0.4%, or at least 0.5% by volume in blood, such as 0.01 to 0.6% or 0.01 to 0.5%.

Disorders Associated with Increased Lactate

The ability to detect NADH/NADPH using the disclosed methods, sensors, and devices permits detection of lactate, and thus permits diagnosis of several disorders, such as tiredness and lactic acidosis. High lactate concentration in the blood is an indicator of tiredness and acidosis. Thus, during exercises or abnormal conditions, monitoring lactate concentration in blood is useful. In addition, lactic acidosis is also the most severe side effect of widely prescribed biguanide drugs for diabetes.⁹ Thus, the disclosed methods and sensors can be used to monitor patients on such drugs. In some examples, the normal concentration range of lactate in blood is about 0.5 to 1 mM, or 0.5 to 22 mM. In some examples, lactic acidosis indicated with values above 5 mM or above 22 mM.

For example, levels of lactate can be determined by monitoring NADH (or NADPH) concentration as compared to a control, such as a reference value of lactate expected in a healthy subject. Thus, in one example, detection of significantly decreased levels of NADH (or NADPH) or no lactate or NADH (or NADPH) (for example relative to a normal control) indicates that the patient from which the sample was obtained has lactic acidosis. For example, if NADH (or NADPH) values are reduced by at least 50%, at least 60%, at least 75%, at least 80%, at least 80%, or at least 95%, relative to lactate or NADH (or NADPH) detected in a sample from a normal individual (such as one without lactic acidosis), this would lead to a diagnosis of lactic acidosis. For example, if lactate values are increased by at least 5%, at least 10%, at least 50%, at least 100%, or at least 200%, relative to lactate detected in a sample from a normal individual (such as one without lactic acidosis), this would lead to a diagnosis of lactic acidosis in the test patient. For example, high lactate in human blood (such as values of >5 mM, such as >22 mM, for example as compared to normal range of 0.5-22 mM) is an indication of high risk in acidosis. Thus, detection NADH or NADPH levels of less than 22 mM in blood can indicate an increased risk of developing acidosis or having lactic acidosis.

Disorders Associated with Increased 3-β-Hydroxybutyrate (3HB)

The ability to detect NADH/NADPH using the disclosed methods, sensors, and devices permits detection of 3HB, and thus permits diagnosis of ketoacidosis, for example in a patient with diabetes (such as type I or type II diabetes). Elevated 3HB concentration in the blood is an indicator of ketoacidosis. Thus, the disclosed methods and sensors can be used to monitor such patients. In some examples, the normal concentration range of 3HB in blood is about 1 mM. In some examples, hyperketonemia is from about 1 mM to 3 mM, and ketoacidosis is above 3 mM.

For example, levels of 3HB can be determined by monitoring NADH (or NADPH) concentration as compared to a control, such as a reference value of 3HB expected in a healthy subject (such as one with a 3HB concentration of about 1 mM). Thus, in one example, detection of significantly increased levels of NADH (or NADPH) (for example relative to a normal control) indicates that the patient from which the sample was obtained has hyperketonemia or ketoacidosis. For example, if NADH (or NADPH) values are increased by at least 50%, at least 60%, at least 75%, at least 80%, at least 80%, or at least 95%, relative to 3HB or NADH (or NADPH) detected in a sample from a normal individual (such as one without hyperketonemia or ketoacidosis), this would lead to a diagnosis of hyperketonemia or ketoacidosis. For example, if 3HB values are increased by at least 20%, at least 50%, at least 100%, at least 200%, or at least at least 300%, relative to 3HB detected in a sample from a normal individual (such as one without hyperketonemia or ketoacidosis), this would lead to a diagnosis of hyperketonemia or ketoacidosis in the test patient.

### Samples

Any specimen that may contain (or is known to contain or is suspected of containing) a target agent can be used. Biological samples are usually obtained from a subject and can include genomic DNA, RNA (including mRNA), protein, metabolites, or combinations thereof. Examples include a tissue or tumor biopsy, fine needle aspirate, bronchioalveolar lavage, pleural fluid, spinal fluid, saliva, sputum, surgical specimen, lymph node fluid, ascites fluid, peripheral blood or fractions thereof (such as serum or plasma), urine, semen, saliva, buccal swab, and autopsy material. Techniques for acquisition of such samples are well known in the art. Serum or other blood fractions can be prepared in the conventional manner. Samples can also include fermentation fluid and tissue culture fluid.

Environmental samples that can be analyzed using the methods provided herein include those obtained from an environmental media, such as water, air, soil, dust, wood, plants or food.

In one example the sample is a food sample that can be analyzed using the methods provided herein, such as a dairy, meat, fruit, or vegetable sample. For example, using the methods provided herein, adulterants in food products can be detected. In one example, an enzyme from a pathogenic bacterium is detected, such as alkaline phosphatase.

In some examples, a sample is a control sample, such as a sample known to contain or not contain a particular amount of the target enzyme or metabolite.

Once a sample has been obtained, the sample can be used directly, concentrated (for example by centrifugation or filtration), purified, liquefied, diluted in a fluid, or combinations thereof. In some examples, the sample is not manipulated prior to its analysis, other than to apply it to the sensor. For example, in some examples the sample is not pre-treated to remove proteins (e.g., hemoglobin or other serum proteins) prior to analysis of the sample. In some examples, the sample is treated to remove glucose (for example by incubating the sample with ATP, MgCl₂, and hexokinase, which in some examples is part of the sensor or device or in other examples this step is done before the sample is contacted with the sensor or device), and the resulting preparation analyzed using the methods provided herein.

### Sensors and Devices for Detecting Target Enzymes or Metabolites

Provided herein are sensors and devices that can be used to detect one or more target enzymes or metabolites. Such sensors and devices can be engineered using the methods provided herein to detect a broad range of targets, significantly facilitating rational design and increasing the efficiency of sensor and device development. Such sensors and devices take advantage of the ability of glucose meters, such as a PGM, to detect coenzymes such as NADH and NADPH.

In some examples, one or more sensors are part of a device, such as a lateral flow device or fluidic device (e.g., microfluidic or macrofluidic device). In some examples, multiple sensors are combined into a single device, thereby permitting detection of more than one target. Using this general methodology, sensitive and selective particular examples for the detection (and in some examples quantification) of G6PD, PK, citrate, lactate, ethanol, and pyruvate are reported herein that require only a commercially available PGM to do the detections. Using this platform, sensors and devices for targets using a PGM can be achieved through the general approach described herein.

Disclosed herein are sensors and devices that permit detection of one or more target agents, such as an enzyme or metabolite, as indicated by an increase or decrease in one or more coenzymes involved in an enzymatic reaction of which the target is a part of. If the enzymatic reaction produces or consumes the coenzyme directly from a starting product (such as G6P), additional enzymes may not be required. However, if the enzymatic reaction produces or consumes the coenzyme indirectly from a starting product (such as PEP), additional enzymes and starting products (e.g., pyruvate and LDH to detect PK) can be supplied (for example with the starting products).

In one example, the sensor includes a solid support to which is attached one or more starting products (such as two or more, three or more, or four or more, such as 1, 2, 3, 4, 5, 6, 7, 8, 9 or 10 different starting products, such as at least one enzyme or substrate and NAD⁺ or NADH, such as at least 2, at least 3, or at least 4, such as 1, 2, 3, 4, 5, 6, 7, 8, 9 or 10 different enzymes or substrates) needed for an enzymatic reaction that utilizes the target enzyme or target metabolite and consumes or generates the coenzyme, which results in coenzyme production or consumption. Methods of attaching or immobilizing reagents to a solid support (such as one that is part of a lateral flow device) are well known in the art. In one example, reagents are suspended (e.g., dissolved) in a solution, such as an aqueous solution, such as water or a buffer, and then applied to the solid support. The solution is then allowed to dry or can be lyophilized, thereby attaching the reagents to the solid support. In some examples the sensor further includes a sample pad, a filter pad, and/or an absorption pad. The sensor can include a solid support having separate areas. In one example, the areas of the sensor having reagents attached thereto are membranes (for example for a lateral flow device), which can in some examples be interspersed with other solid supports without reagents (such as other membranes). In another example, the areas of the sensor having reagents attached thereto are beads (e.g. magnetic beads or polymer beads, such as agarose or sepharose beads), or walls of a chamber (for example for a micro- or macro-fluidic device), which can in some examples be interspersed with other solid supports without reagents (such as one or more filters).

In one example, the sensor includes a solid support which includes one or more discrete regions having attached thereto one or more starting products needed for an enzymatic reaction that utilizes the target enzyme or target metabolite and consumes or generates the coenzyme, which results in coenzyme production or consumption. Such a solid support can form one or more walls of a chamber (e.g., a holding chamber or a mixing chamber were desired reactions can occur) that is part of the sensor or device. For example, the device can include a first (and optionally a second) mixing chamber, which can include buffers, pre-treatment reagents, and/or one or more starting products (such as two or more, three or more, or four or more, such as 1, 2, 3, 4, 5, 6, 7, 8, 9 or 10 different starting products, such as at least one enzyme or substrate and NAD⁺ or NADH), between the region containing the sample port and the region containing the outlet. The sensor can include a solid support having separate areas. In one example, the areas of the sensor having reagents associated therewith are holding or mixing chambers, which can in some examples be interspersed with other areas (such as a filter). In some examples, such as a lateral flow device, the solid support includes one or more membranes.

In some examples, the solid support includes a region (such as a membrane or chamber) that has attached thereto reagents that can neutralize a basic pH to a neutral pH, such as a phosphate buffer (e.g., sodium, potassium or ammonium salts, for example, NaH₂PO₄ and the like), as well as other buffers such as Tris-HCl, HEPES, MES, NaHCO₃—Na₂CO₃, and the like. Such a region is generally located on the device or sensor after regions where the enzymatic reaction takes place (e.g., generation or consumption of coenzyme).

A solid support can also have an entry and exit port. In one example, the solid support includes a region (such as a membrane or chamber) that includes buffer reagents (such as Tris-HCl, HEPES, MES, NaHCO₃—Na₂CO₃, and the like). The coenzyme can be released from the device, for example through an exit port, wherein it is subsequently detected and in some examples quantified, using a PGM. In some examples, the solid support includes a region (such as a membrane or chamber) that has attached thereto reagents that can neutralize a basic pH to a neutral pH, such as a phosphate buffer (e.g., sodium, potassium or ammonium salts, for example, NaH₂PO₄ and the like), as well as other buffers such as Tris-HCl, HEPES, MES, NaHCO₃—Na₂CO₃, and the like. Such a region is generally before the exit port, and after regions where the enzymatic reaction takes place (e.g., generation or consumption of coenzyme).

The coenzyme can be detected and in some examples quantified, using a PGM. In some examples, the amount of coenzyme produced or consumed is proportional to the amount of target enzyme or target metabolite in the test sample. Thus, in some examples, if coenzyme is detected, this indicates that the target or is present in the sample. In other examples, if coenzyme is not detected or is reduced, this indicates that the target is not present or is decreased in the sample (relative to a normal sample).

In one example, the target enzyme is G6PD. In such an example, the one or more starting products include G6P and NAD+ or NADP+. Thus, the solid support can include an area or region that has attached thereto, or holds, G6P and NAD+ or NADP+.

In another example, the target enzyme is PK. In such an example, the one or more starting products include phosphoenolpyruvate (PEP), adenosine diphosphate (ADP), NADH and lactate dehydrogenase (LDH). Thus, the solid support can include an area or region that has attached thereto, or holds, PEP, ADP, NADH and LDH.

In one example, the target metabolite is citrate. In such an example, the one or more starting products include citrate lyase (CL), acetyl coenzyme A, malate dehydrogenase (MDH), NADH and LDH. Thus, the solid support can include an area or region that has attached thereto, or holds, CL, acetyl coenzyme A, MDH, NADH and LDH.

In another example, the target metabolite is lactate. In such an example, the one or more starting products include lactate oxidase (LOx), LDH and NADH. Thus, the solid support can include an area or region that has attached thereto, or holds, LOx, LDH and NADH.

In one example, the target metabolite is ethanol. In such an example, the one or more starting products include ADH and NAD⁺. Thus, the solid support can include an area or region that has attached thereto, or holds, ADH and NAD⁺.

In one example, the target metabolite is 3-β-hydroxybutyrate (3HB). In such an example, the one or more starting products include 3-hydroxybutyrate dehydrogenase (HBDH) and NAD⁺. Thus, the solid support can include an area or region that has attached thereto, or holds, HBDH and NAD⁺.

In one example, the target metabolite is pyruvate. In such an example, the one or more starting products include LDH, LOx, and NADH. Thus, the solid support can include an area or region that has attached thereto, or holds, LDH, LOx, and NADH. Required oxygen for the reaction can be supplied from the ambient air.

In one example, the target metabolite is creatinine. In such an example, the one or more starting products include creatininase, ATP, creatine kinase, PK, PEP, LDH, LOx, and NADH. Thus, the solid support can include an area or region that has attached thereto, or holds, creatininase, ATP, creatine kinase, PK, PEP, LDH, LOx, and NADH. Required oxygen for the reaction can be supplied from the ambient air.

In another example, the target metabolite is FAD-dependent D-lactate dehydrogenase and the one or more starting products include FADH₂. Thus, the solid support can include an area or region that has attached thereto, or holds, FADH₂.

In another example, the target metabolite is FAD-dependent sarcosine oxidase and the one or more starting products comprise FAD. Thus, the solid support can include an area or region that has attached thereto, or holds, FAD.

In another example, the target metabolite is FAD-dependent D-proline dehydrogenase and the one or more starting products comprise FAD. Thus, the solid support can include an area or region that has attached thereto, or holds, FAD.

In another example, the target metabolite is FMN-dependent L-lactate dehydrogenase and the one or more starting products comprise FMNH₂. Thus, the solid support can include an area or region that has attached thereto, or holds, FMNH₂.

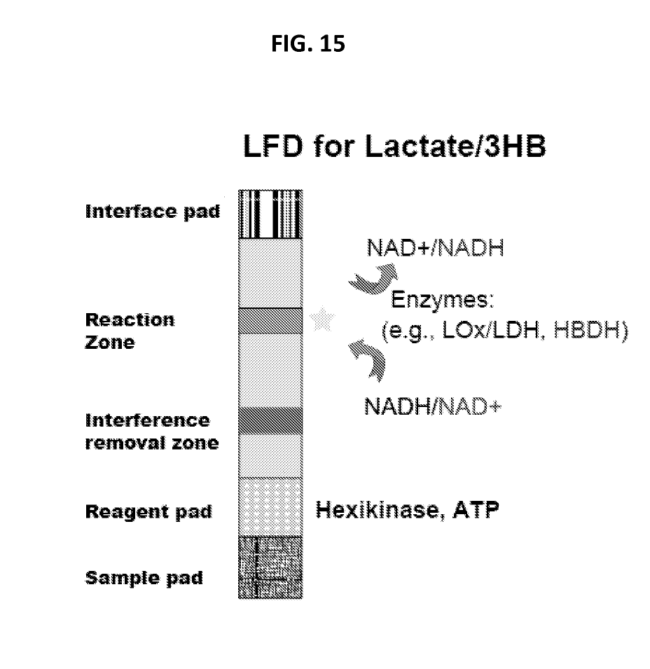

In some examples, in addition to containing one or more starting products, the solid support can include an area or region that has attached thereto, or holds, reagents to remove glucose from the sample, such as hexokinase (and optionally MgCl₂ and/or ATP). In one example, such glucose-removing reagents are suspended (e.g., dissolved) in a solution, such as an aqueous solution, such as water or a buffer, and then applied to the solid support. The solution is then allowed to dry or can be lyophilized, thereby attaching the reagents to the solid support. In some examples, such agents are on the same solid support as the starting products. In other examples, such agents are in/on a different region of the solid support, such as a region that is encountered by the sample prior to the region containing the starting products (e.g., see FIG. 15).

The solid support can further include attached thereto agents that permit detection of a coenzyme, such as materials routinely found on commercially available glucose meter strips. For example, the “endpoint” of the disclosed solid supports (for example an area containing a coenzyme or from where a coenzyme is released) can be connected to the start point of any commercial glucose meter strip (such as those containing glucose oxidase or glucose dehydrogenase). Exemplary glucose meter strips include but are not limited to Glucocard® and OneTouch® (also see U.S. Pat. Nos. 6,413,410 and 6,733,655). Thus, in one example, the solid support further includes a region that has attached thereto glucose oxidase.

The sensor can include additional materials, such as one or more reaction pads (or membranes) on which the desired reactions can occur, a sample pad to which the sample is applied, a filter to remove or reduce unnecessary materials (such as cell debris or large proteins), an absorption pad where the cofactor or coenzyme is delivered and read by a glucose meter, or combinations thereof.

Methods of using the sensors (such as a lateral flow device, macrofluidic device or microfluidic device) for one or more targets are also provided. For example, such a method can include contacting a sensor with a test sample under conditions sufficient to allow a target in the test sample to interact with the one or more starting products, under conditions wherein a coenzyme is produced or consumed. In some examples, this reaction occurs on a reaction pad, such as a first or second reaction pad. In some examples, this reaction occurs in a mixing or holding chamber. The coenzyme is detected, for example with a PGM. In some examples, the coenzyme is present on an absorption pad, which is read by a PGM. The amount of coenzyme detected can be proportional or inversely proportional to the amount of target present in the sample. Thus, in some examples the method includes determining that the target agent is present in the sample when the coenzyme is detected, while in other examples the method includes determining that the target agent is not present in the sample when the coenzyme is not detected.

In a specific example, the method includes contacting one or more lateral flow devices with a sample under conditions sufficient to allow the target in the sample to flow through the lateral flow device and interact with the one or more starting products present on the lateral flow device (for example on a first reagent pad), and generating or consuming a coenzyme (for example on a first reaction pad). If present, the coenzyme can flow to an absorption pad present on the lateral flow device. In some examples, the coenzyme flows through the lateral flow device and interacts with pH neutralizing agents present on the lateral flow device. The coenzyme is detected, for example with a PGM. In some examples, the coenzyme is present on an absorption pad, which is read by a PGM.

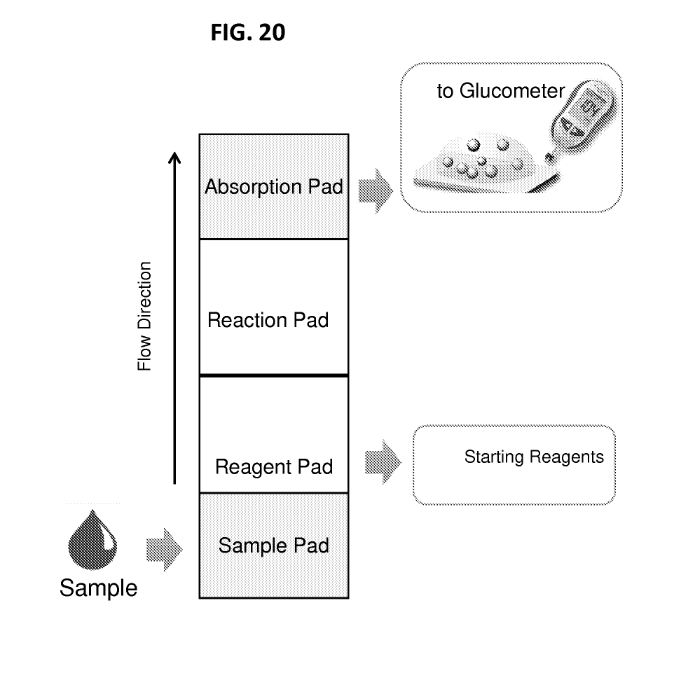

For example, as shown in FIG. 20, samples containing target or not are applied to the sample pad of a lateral flow device. If desired, liquid can be added to the sample, or the sample can be concentrated, before applying it to the sample pad. In one example, a fluid sample containing or suspected of containing a target is applied to the sample pad, for example dropwise or by dipping the end of the device into the sample. If the sample is whole blood, an optional developer fluid can be added to the blood sample to cause hemolysis of the red blood cells and, in some cases, to make an appropriate dilution of the whole blood sample. In some examples wherein the sample is whole blood, the sample pad can include one or more reagents that result in lysis of the red blood cells, such as a sample pad containing ammonium chloride, potassium bicarbonate and EDTA. In some examples (not shown), the sample pad (or subsequent reagent pad, or the reagent pad containing the starting reagents) includes hexokinase (and optionally ATP and/or MgCl₂) to remove glucose in the sample. The sample pad ensures a controllable (unilateral) flow of the sample. The sample migrates from the bottom to the top of the lateral flow device following the indicated flow direction in FIG. 20 because of capillary force. In the reagent pad, the solution can take starting reagents present in the reagent pad along with it and then they reach the higher reaction pad. In other examples (not shown), the solution migrates to the starting reagents present in the reagent pad, where the reaction is allowed to take place, and the resulting products migrate to a subsequent absorption pad. In one example, reagents are suspended (e.g., dissolved) in a solution, such as an aqueous solution, such as water or a buffer, and then applied to the solid support. The solution is then allowed to dry or can be lyophilized, thereby attaching the reagents to the solid support (such as a reagent pad). Exemplary starting reagents are provided herein, and will depend on the target. In some examples, the reagent pad includes hexokinase, ATP and MgCl₂ to remove glucose in the sample. At the reaction pad, target in the sample and the starting products can react to produce or consume a coenzyme. However, in some examples, a reaction pad is not needed, and instead the reaction occurs in the reagent pad. An optional second reagent pad includes one or more agents that can neutralize a pH from basic to neutral (such as an acidic buffer or reagents). Coenzymes or cofactors will ultimately flow to the absorption pad, which can be read by a PGM. Such a lateral flow device can include a bibulous lateral flow strip, which can be present in housing material (such as plastic or other material). The amount of coenzyme detected can be proportional or inversely proportional to the amount of target present in the sample. Thus, in some examples the method includes determining that the target agent is present in the sample when the cofactor or coenzyme is detected, while in other examples the method includes determining that the target agent is not present in the sample when the coenzyme or cofactor is not detected.

Another example of an exemplary lateral flow device for detecting lactate or 3HB is shown in FIG. 15. Samples containing target or not are applied to the sample pad. If desired, liquid can be added to the sample, or the sample can be concentrated, before applying it to the sample pad. In one example, a fluid sample containing or suspected of containing a target is applied to the sample pad, for example dropwise or by dipping the end of the device into the sample. If the sample is whole blood, an optional developer fluid can be added to the blood sample to cause hemolysis of the red blood cells and, in some cases, to make an appropriate dilution of the whole blood sample. In some examples wherein the sample is whole blood, the sample pad can include one or more reagents that result in lysis of the red blood cells, such as a sample pad containing ammonium chloride, potassium bicarbonate and EDTA. In the examples shown in FIG. 15, the sample travels to a first reagent pad containing hexokinase (and optionally ATP and/or MgCl₂), which allows glucose in the sample to be removed. The sample then travels to a second reagent pad (and/or reaction zone) containing the starting products, such as NADH, LOx, and LDH (to detect lactate) or NAD⁺ and HBDH to detect 3HB. In the reagent pad or the reaction zone, the NAD⁺ or NADH is produced, which will ultimately flow to the interface (absorption pad), which can be read by a PGM. Such a lateral flow device can include a bibulous lateral flow strip, which can be present in housing material (such as plastic or other material). The amount of NAD⁺ or NADH detected can be proportional or inversely proportional to the amount of lactate or 3HB present in the sample. Thus, in some examples the method includes determining that the 3HB is present in the sample when NADH is detected, while in other examples the method includes determining that the lactate is not present in the sample when NADH is not detected. One skilled in the art will appreciate that similar lateral flow devices can be made to detect other target metabolites or enzymes using the teachings herein.

In another specific example, the method includes contacting one or more fluidic devices with a sample under conditions sufficient to allow the target agent in the sample to flow through the device and interact with the one or more starting products present in the fluidic device (macro- or micro-). For example, starting products can be present in a single holding or mixing chamber, or divided among chambers. In some examples, in or more starting products can be introduced into the device (e.g., allowed to flow into the device). The enzymatic reaction is allowed to proceed, for example in a mixing or holding chamber, generating or consuming a coenzyme. If present, the coenzyme can flow through the device, for example through an exit port on the macro- or micro-fluidic device. In some examples, the coenzyme flows through the macro- or micro-fluidic device and interacts with pH neutralizing agents present in the macro- or micro-fluidic device (such as in a second holding or mixing chamber). The coenzyme is detected, for example with a PGM.

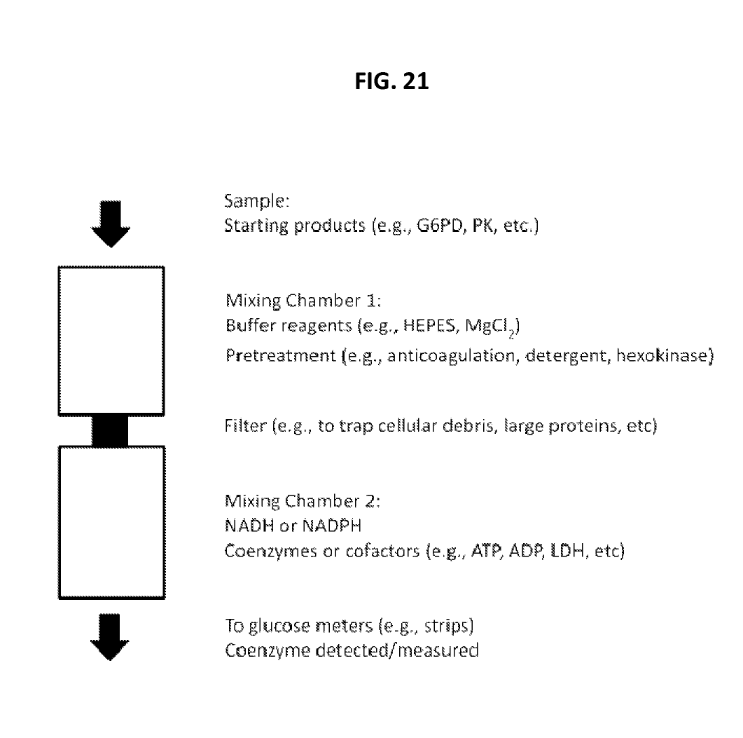

An exemplary microfluidic device that can be used with the methods provided herein is shown in FIG. 21. The microfluidic device controls the movement of the sample and other liquids, dispenses reagents, and merges or splits a micro-size droplet in the microfluidic device via the voltage applied to the flow versus the device. As shown in FIG. 21, the device includes a sample entry port at the top, where the test sample is introduced into the device. In some examples, the starting enzyme (e.g., G6P) can be added to the sample before it is introduced into the device. The solution flows from the top to the bottom. The device also includes an area containing one or more holding or mixing chambers, which can include one or more of the starting products, reagents needed to remove glucose from the sample (such as hexokinase, MgCl₂, and ATP), red blood cell lysis buffers, anti-coagulation agents, detergents, suitable buffers for the enzymatic reaction, buffers to neutralize the reaction, or combinations thereof. Such holding or mixing chambers can dispense the reagents therein, for example in the form of a droplet, or hold or contain the reagents. As shown in FIG. 21, the device includes one or more mixing chambers, where desired reactions can occur (such as the removal of glucose from a sample, production or consumption of cofactors and coenzymes, or combinations thereof). The sample is introduced into the entry port. In some examples the starting enzyme is added at this point. However, one skilled in the art will appreciate that the starting enzyme can be present in a mixing or holding chamber instead. The sample travels to a first mixing or holding chamber containing buffers and pre-treatment reagents, such as those needed to remove glucose, lyse cells, anticoagulation agents, detergents, or combinations thereof. In some examples, such agents are divided over more than one chamber. The sample can then flow through a filter, for example to remove cellular debris and large proteins. Such a filter can be a membrane or other suitable material. The sample then flows into a second mixing or holding chamber containing the additional reagents needed for the enzymatic reaction (e.g., NADP+, NAD+, ATP, ADP and in some examples the starting enzyme). The sample and reagents are incubated in the second mixing chamber for sufficient time to ensure completion of the enzymatic reaction (e.g., production or consumption of the coenzyme). In some examples, the device also includes a region following of the second mixing or holding chamber, such as a third holding or mixing chamber (not shown) for buffers and/or neutralizing reagents, that can for example neutralize the pH of the reaction (e.g., droplet) released from the second mixing or holding chamber. Thus, the sample moves through successive chamber(s) taking with it reagents therein, such as starting reagents, which allows target in the sample (if present) and the starting products to react can produce or consume a coenzyme. Produced cofactors or coenzymes will flow to the exit port, which can be read by a PGM.

Although a particular configuration is shown in FIG. 21, one skilled in the art will appreciate that other configurations are possible, for example more regions or mixing chambers if multiple targets are to be detected in the same sample on the same device. For example the device can have discrete regions and mixing chambers for each target to be detected. In such an example, the device may include multiple exit ports, one for each target. The amount of coenzyme detected can be proportional or inversely proportional to the amount of target present in the sample. Thus, in some examples the method includes determining that the target agent is present in the sample when the coenzyme is detected, while in other examples the method includes determining that the target agent is not present in the sample when the coenzyme is not detected.

### Solid Supports

The solid support which forms the foundation of the sensor (or device) can be formed from known materials, such as any water immiscible material. In some examples, suitable characteristics of the material that can be used to form the solid support surface include: being amenable to surface activation such that upon activation, the surface of the support is capable of covalently attaching desired reagents, such as starting products (e.g., enzymes, substrates, ATP, ADP, MgCl₂, NAD+, NADP+ and the like); being chemically inert such that at the areas on the support not occupied by the desired reagents can interact with the target with desired specificity are not amenable to non-specific reactions or binding, or when non-specific binding or reactions occurs, such materials can be readily removed from the surface without removing the desired reagents or target agent. In some examples, the solid support includes a plurality of materials, such as starting products immobilized onto beads, wherein the beads are immobilized onto a membrane (such as a reagent pad).

A solid phase can be chosen for its intrinsic ability to attract and immobilize an agent, such as starting products. The factor can include a charged substance that is oppositely charged with respect to, for example, the desired reagent or to a charged substance conjugated to the desired reagent. Antibodies and aptamers can be used to attach desired proteins.

The surface of a solid support may be activated by chemical processes that cause covalent linkage of a desired reagent (e.g., enzymes, substrates, ATP, ADP, MgCl₂, NAD⁺, NADP⁺ and the like) to the support. However, any other suitable method may be used for immobilizing a desired reagent to a solid support including, without limitation, ionic interactions, hydrophobic interactions, covalent interactions and the like. The particular forces that result in immobilization of a desired reagent on a solid phase are not important for the methods and devices described herein.

In one example the solid support includes a particle, such as a bead. Such particles can be composed of metal (e.g., gold, silver, platinum), metal compound particles (e.g., zinc oxide, zinc sulfide, copper sulfide, cadmium sulfide), non-metal compound (e.g., silica or a polymer such as agarose or sepharose), as well as magnetic particles (e.g., iron oxide, manganese oxide). In some examples the bead is a latex or glass bead. The size of the bead is not critical; exemplary sizes include 5 nm to 5000 nm in diameter. In one example such particles are about 1 μm in diameter.

In another example, the solid support is a bulk material, such as a paper, membrane, porous material, water immiscible gel, water immiscible ionic liquid, water immiscible polymer (such as an organic polymer), and the like. For example, the solid support can include a membrane, such as a semi-porous membrane that allows some materials to pass while others are trapped. In one example the membrane comprises nitrocellulose. In a specific example the solid support is part of a lateral flow device that includes one or more regions containing the sensors disclosed herein. In a specific example the solid support is part of a macro- or micro-fluidic device that includes one or more regions containing the sensors disclosed herein. For example, a fluidic device can include one or more chambers holding or having attached thereto desired reagents. In some examples such chambers are made of plastics or other polymer materials, which are well known in the art.

In some embodiments, porous solid supports, such as nitrocellulose, are in the form of sheets or strips, such as those found in a lateral flow device. The thickness of such sheets or strips may vary within wide limits, for example, at least 0.01 mm, at least 0.1 mm, or at least 1 mm, for example from about 0.01 to 5 mm, about 0.01 to 2 mm, about 0.01 to 1 mm, about 0.01 to 0.5 mm, about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

In one example, the solid support is composed of an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof).

In yet other examples, the solid support is a material containing, such as a coating containing, any one or more of or a mixture of the ingredients provided herein.

A wide variety of solid supports can be employed in accordance with the present disclosure. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

The solid support can be any format to which the molecule specific for the test agent can be affixed, such as microtiter plates, ELISA plates, test tubes, inorganic sheets, dipsticks, lateral flow devices, and the like. One example includes a linear array of molecules specific for the target agent, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range.

In one example the format is a bead, such as a silica bead. In another example the format is a nitrocellulose membrane. In another example the format is filter paper. In yet another example the format is a glass slide. In one example, the solid support is a polypropylene thread. One or more polypropylene threads can be affixed to a plastic dipstick-type device; polypropylene membranes can be affixed to glass slides.

In one example the solid support is a microtiter plate. For example sensors can be affixed to the wells of a microtiter plate (for example wherein some wells can contain a sensor to detect target X, while other wells can contain a sensor to detect target Y; or several wells might include the same sensor, wherein multiple samples can be analyzed simultaneously). The test sample potentially containing a target of interest can be placed in the wells of a microtiter plate containing a sensor disclosed herein, and the presence of the target detected using the methods provided herein in. The microtiter plate format permits testing multiple samples simultaneously (together with controls) each in one or more different wells of the same plate; thus, permitting high-throughput analysis of numerous samples.

In some examples, a disclosed sensor is attached to more than one solid support. For example, a sensor containing starting products specific for the target can be attached to a bead, which can then be attached to a conjugation or reagent pad of a lateral flow device.

Each of the supports and devices discussed herein (e.g., microfluidic device, lateral flow device) can be, in some embodiments, formatted to detect multiple targets by the addition of reagents specific for the other targets of interest. For example, certain wells of a microtiter plate can include molecules specific for the other targets of interest. Some flow device embodiments can include secondary, tertiary or more capture areas containing molecules specific for the other targets of interest.

### Attaching Materials to a Solid Support

Methods of conjugating a desired reagent (e.g., starting products such as enzymes, substrates, ATP, ADP, MgCl₂, NAD⁺, NADP⁺ and the like) to the solid support (such as a reagent pad or to a holding chamber) are conventional. The conjugation method used can be any chemistry that can covalently or non-covalently incorporate a desired reagent with other molecules. In some examples, a desired reagent (e.g., enzymes, substrates, ATP, ADP, MgCl₂, NAD⁺, NADP⁺ and the like) is attached to a solid support, such as a conjugation pad of a lateral flow device or a holding chamber of a flow device, simply by suspending the reagent in a solution, applying the solution to the pad, and allowing the solution to dry.

In one example the method uses a reaction that forms covalent bonds including but not limited to those between amines and isothiocyanates, between amines and esters, between amines and carboxyls, between thiols and maleimides, between thiols and thiols, between azides and alkynes, and between azides and nitriles. In another example, the method uses a reaction that forms non covalent interactions including but not limited to those between antibodies and antigens, and between organic chelators and metal ions.

### Lateral Flow Devices

In one example, the solid support is a lateral flow device, which can be used to determine the presence and/or amount of one or more targets in a sample, such as a liquid sample. A lateral flow device is an analytical device having a test strip, through which flows a test sample fluid that is suspected of (or known to) containing a target. Lateral flow devices are useful to simplify and automate user sample interface and processing. One example of a lateral flow device is a glucose testing strip. Based on the principles of a glucose testing or pregnancy strip, lateral flow devices that incorporate the disclosed sensors can be developed. In some examples, by using such as lateral flow devices, samples can be directly contacted with or applied to the lateral flow device, and no further liquid transfer or mixing is required. Such devices can be used to detect target agents, for example qualitatively or quantitatively.

Lateral flow devices are commonly known in the art, and have a wide variety of physical formats. Any physical format that supports and/or houses the basic components of a lateral flow device in the proper function relationship is contemplated by this disclosure. In one example, the lateral flow devices disclosed in U.S. Pat. No. 7,799,554, Liu et al. (Angew. Chem. Int. Ed. 45:7955-59, 2006), Apilux et al. (Anal. Chem. 82:1727-32, 2010), Dungchai et al. (Anal. Chem. 81:5821-6, 2009), or Dungchai et al. (Analytica Chemica Acta 674:227-33, 2010) (all herein incorporated by reference) are used, such as one made using the Millipore Hi-Flow Plus Assembly Kit. There are a number of commercially available lateral flow type tests and patents disclosing methods for the detection of large analytes (MW greater than 1,000 Daltons) (see for example U.S. Pat. Nos. 5,229,073; 5,591,645; 4,168,146; 4,366,241; 4,855,240; 4,861,711; and 5,120,643; European Patent No. 0296724; WO 97/06439; and WO 98/36278). There are also lateral flow type tests for the detection of small-analytes (MW 100-1,000 Daltons) (see for example U.S. Pat. Nos. 4,703,017; 5,451,504; 5,451,507; 5,798,273; and 6,001,658). There are also lateral flow type tests for the detection of glucose (see for example U.S. Pat. Nos. 6,413,410 and 6,733,655).

The construction and design of lateral flow devices is very well known in the art, as described, for example, in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Lateral flow devices generally include a strip of absorbent material (such as a microporous membrane), which can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the reagents (such as starting products or antibodies) required for the detection and/or quantification of the particular target being tested for. Thus these zones can be viewed as functional sectors or functional regions within the test device.

These devices typically include a sample application area and one or more separate reagent areas in which desired reagents are immobilized (such as a sensor disclosed herein). For example, a lateral flow device containing at least two separate reagent areas (such as 2, 3, 4, 5, 6, 7, 8, 9, 10, 11, 12, 13, 14, 15 or more) can be used to detect a plurality of different targets in a single sample. Any liquid (such as a fluid biological sample) applied in the sample application area flows along a path of flow from the sample application area, through the reagent area(s), through an optional reaction area, to the absorption area. Ultimately, coenzymes, if produced, flow to a downstream absorbent pad, which can act as a liquid reservoir. The resulting coenzyme on the lateral flow strip can be detected with a PGM, for example by insertion of the device into a PGM.

In one example where a lateral flow device can detect multiple targets, the device includes a single wicking pad or sample application area, and multiple conjugation or reagent pads, membranes or reaction pads, and absorption pads (such that one or more conjugation pads are associated with one or more particular membranes and an absorption pad). For example, each conjugation pad(s) can include a different reagents needed to detect a particular target. Thus, the glucose produced as a result of the target and present on each absorption pad can be used to detect the presence of a particular target.

The lateral flow device can include a wicking or sample pad (e.g., made of glass fiber, woven fibers, screen, non-woven fibers, cellulosic filters, or paper), reagent pad (e.g., made of glass fiber, polyester, paper or surface modified polypropylene), reaction pad (e.g., made of nitrocellulose (including pure nitrocellulose and modified nitrocellulose), nitrocellulose direct cast on polyester support, polyvinylidene fluoride, or nylon), absorption pad (e.g., made of cellulosic filters or paper), and combinations thereof. Such pads can abut one another or overlap, and can be attached to a backing. One of skill in the art will recognize that the particular materials used in a particular lateral flow device will depend on a number of variables, including, for example, the target to be detected, the sample volume, the desired flow rate and others, and can routinely select the useful materials accordingly.

The test sample is applied to or contacted with the sample pad (which is usually at the proximal end of the device, but can for example be at the center of the device for example when multiple reagent pads are included to detect multiple targets), for instance by dipping or spotting. A sample is collected or obtained using methods well known to those skilled in the art. The test sample may be obtained from any biological, environmental, or food source. In some examples, the sample is diluted, purified, concentrated, filtered, dissolved, suspended or otherwise manipulated prior to assay. The fluid sample migrates distally through all the functional regions of the strip. The final distribution of the fluid in the individual functional regions depends on the adsorptive capacity and the dimensions of the materials used.

The sample pad ensures that the sample moves through the device in a controllable manner, such that it flows in a unilateral direction. The sample pad initially receives the sample, and can serve to remove particulates from the sample. Among the various materials that can be used to construct a sample pad, a cellulose sample pad may be beneficial if a large bed volume (e.g., 250 μl/cm²) is a factor in a particular application. In one example, the sample pad is made of Millipore cellulose fiber sample pads (such as a 10 to 25 mm pad, such as a 15 mm pad). Sample pads may be treated with one or more release agents, such as buffers, salts, proteins, detergents, and surfactants. Such release agents may be useful, for example, to promote resolubilization of reagent-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%). In one example the sample pad includes reagents to lyse RBCs, such as one containing ammonium chloride, potassium bicarbonate and EDTA, such ammonium chloride (8.26 g), potassium bicarbonate (1 g) and EDTA (0.037 g) in 1 liter water (0.037 g) in 1 liter water.

After contacting the sample to the sample pad, the sample liquid migrates from bottom to the top because of capillary force (or from the center outwards). The sample then flows to a first reagent pad, which serves to, among other things, hold at least some of the reagents needed to convert the target into a corresponding coenzyme signal. The reagents can be immobilized to the reagent pad by spotting (for example the starting reagents can be suspended in water or other suitable buffer and spotted onto the conjugation pad and allowed to dry). In one example the reagent pad is 10 to 25 mm, for example 13 mm. When the sample reaches the reagent pad, target agent present in the sample can interact with the reagents immobilized to the reagent pad. In some examples, reactions, such as binding reactions, occur on reaction pads. In some examples, the sample passes through a series of reagent and reaction/membrane pads. The reaction pad can be made of known materials, such as a HiFlow Plus Cellulose Ester Membrane, such as one that is 10 to 40 mm, for example 25 mm. Finally, if a c coenzyme is produced, it moves with the flow and reaches the absorption pad, where it is then detected by a PGM (for example it can be attached to a commercially available glucose strip, such as one containing glucose oxidase). The absorbent pad acts to draw the sample across the reagent pad(s) and membrane(s) by capillary action and collect it. This action is useful to insure the sample solution will flow from the sample pad unidirectionally through reagent pad to the absorption pad. Any of a variety of materials is useful to prepare an absorbent pad. In some embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent pad made may be adjusted by changing the dimensions (usually the length) of an absorbent pad. In one example the absorption pad is 10 to 25 mm, for example 15 mm.

### Fluidic Devices

In one example, the solid support is a macro- or micro-fluidic device, which can be used to determine the presence and/or amount of one or more target agents in a sample, such as a liquid sample. Such devices are also referred to as “lab-on-a-chip” devices. The development of microfluidics and microfluidic techniques has provided improved chemical and biological research tools, including platforms for performing chemical reactions, combining and separating fluids, diluting samples, and generating gradients (for example, see U.S. Pat. No. 6,645,432 and WO 2010091080).

A portable macro- or micro-fluidic device can be transported to almost any location. For macro- or microfluidic assays and devices, test samples (such as a liquid sample) can be supplied by an operator, for example using a micropipette. The sample to be tested may be obtained from any biological source. In some examples, the sample is diluted, purified, concentrated, filtered, dissolved, suspended or otherwise manipulated prior to assay. A test sample can be introduced into an inlet of a microfluidic system and the fluid may be drawn through the system, for example by application of a vacuum source to the outlet end of the macro- or micro-fluidic system.

In some examples, starting reagents are pumped in, for instance by using different syringe pumps filled with the required reagents. After one fluid is pumped into the microfluidic device, a second can be pumped in by disconnecting a line from the first pump and connecting a line from a second pump. Alternatively, valving may be used to switch from one pumped fluid to another. Different pumps can be used for each fluid to avoid cross contamination, for example when two fluids contain components that may react with each other or, when mixed, can affect the results of an assay or reaction. Continuous flow systems can use a series of two different fluids passing serially through a reaction channel. Fluids can be pumped into a channel in serial fashion by switching, through valving, the fluid source that is feeding the tube. The fluids constantly move through the system in sequence and are allowed to react in the channel.

As an alternative (or in addition to) introducing starting reagents into the fluidic device, starting reagents and other desired materials can be present in one or more mixing or holding chambers, and the sample allowed to interact with reagents in the chambers as it passes through the device.

Macro- and micro-fluidic devices for analyzing a target analyze are well known, and can be adapted using the disclosed system to detect a target of interest. For example devices from Axis Shield (Scotland), such as the Afinion analyzer, analyzers from Claros (Woburn, Mass.), and devices from Advanced Liquid Logic (Morrisville, N.C.) such as those based on eletrowetting. Other exemplary devices are described in US Patent Publication Nos. 20110315229; 20100279310; 2012001830 and 2009031171.

### Kits

The disclosure also provides kits that include one or more of the sensors or devices disclosed herein, for example sensors that are part of a lateral flow or fluidic device. For example, a kit can include at least 2 different sensors permitting detection of at least two different target agents, such as at least 3, at least 4, at least 5, or at least 10 different sensors. In a specific example, a kit can include at least 2 different microfluidic or macrofluidic devices permitting detection of at least two different target agents, such as at least 3, at least 4, at least 5, or at least 10 different microfluidic or macrofluidic devices. In another specific example, a kit can include at least 2 different lateral flow devices permitting detection of at least two different target agents, such as at least 3, at least 4, at least 5, or at least 10 different lateral flow devices.

The kits can include one or more sensors or devices and a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes one or more containers, for instance vials, tubes, and the like that can retain, for example, one or more biological samples to be tested, positive and/or negative control samples or solutions (such as, a positive control sample containing the target agent), diluents (such as, phosphate buffers, or saline buffers), a PGM, and/or wash solutions (such as, Tris buffers, saline buffer, or distilled water).

Such kits can include other components, such as a buffer, a chart for correlating detected coenzyme level and amount of target present, the reagents needed to ultimately produce or consume the coenzyme (such as ATP, enzymes, and NAD+) or combinations thereof. For example, the kit can include a vial containing one or more of the sensors or devices disclosed herein and a separate vial containing a buffer (for example a lysis buffer).

Other kit embodiments include syringes, finger-prick devices, alcohol swabs, gauze squares, cotton balls, bandages, latex gloves, incubation trays with variable numbers of troughs, adhesive plate sealers, data reporting sheets, which may be useful for handling, collecting and/or processing a biological sample. Kits may also optionally contain implements useful for introducing samples onto or into a device, including, for example, droppers, Dispo-pipettes, capillary tubes, rubber bulbs (e.g., for capillary tubes), and the like. Still other kit embodiments may include disposal means for discarding a used device and/or other items used with the device (such as patient samples, etc.). Such disposal means can include, without limitation, containers that are capable of containing leakage from discarded materials, such as plastic, metal or other impermeable bags, boxes or containers.

In some examples, a kit will include instructions for the use of a sensor or device disclosed herein. The instructions may provide direction on how to apply sample to the sensor or device, the amount of time necessary or advisable to wait for results to develop, and details on how to read and interpret the results of the test. Such instructions may also include standards, such as standard tables, graphs, or pictures for comparison of the results of a test. These standards may optionally include the information necessary to quantify target using the sensor or device, such as a standard curve relating amount of coenzyme detected to an amount of target therefore present in the sample.

### Example 1

### Materials and Methods

This example describes the materials and methods used in Examples 2-9 below.

### Materials

All the reagents were purchased from Sigma-Aldrich (St. Louis, Mo., USA). The tests were carried out in HEPES Buffer (200 mM HEPES buffer pH 7.5, 4 mM MgCl₂, 50 mM KCl), horse blood, calf blood, human blood, or human serum. Personal glucose meter (PGM) measurements were applied to the sample solutions mixed with suitable substrates or enzymes after a defined time as indicated in the following procedures. The PGM used was a Bayer Breeze 2® glucose meter. Other glucose meters can also be used instead due to their response to NADH.

### Glucose-6-Phosphate Dehydrogenase (G6PD) Assay

In a typical test, 80 μL HEPES Buffer or human serum was spiked by 1 μL G6PD stock solution to a final concentration of 0-3 U/mL G6PD to serve as test samples. Then 10 μL 600 mM glucose-6-phosphate and 10 μL 250 mM NAD+(or NADP+) in HEPES Buffer were added to the above samples to initiate the enzymatic reaction (final concentrations of the reagents were 60 mM glucose-6-phosphate and 25 mM NAD+(or NADP+)). After a defined time (4, 6, or 24 min), the solution was tested by a PGM.

### Pyruvate Kinase (PK) Assay

In a typical test, 80 μL human serum was spiked by 1 μL PK stock solution to a final concentration of 0 to 6 U/mL PK to serve as test samples. Then 5 μL 1.2 M phosphoenolpyruvate (PEP), 10 μL 600 mM ADP, 6 μL 250 mM NADH and 1 μL 10 kU/mL lactate dehydrogenase (LDH) in HEPES Buffer were added to the above samples to initiate the coupled enzymatic reactions (final concentration of the reagents were 60 mM PEP, 60 mM ADP, 15 mM NADH and 100 U/mL LDH). After 10 min, the solution was tested by a PGM.

### Citrate Assay

In a typical test, 54 μL HEPES Buffer was spiked by 6 μL citrate stock solution to a final concentration of 0-12 mM citrate to serve as test samples. Then 10 μL 50 U/mL citrate lyase (CL) containing 1 mM acetyl coenzyme A, 10 μL 100 U/mL malate dehydrogenase (MDH), 20 μL 75 mM NADH and 1 μL 10 kU/mL lactate dehydrogenase (LDH) in HEPES Buffer were added to the above samples to initiate the coupled enzymatic reactions (final concentration of the reagents were 5 U/mL CL, 10 U/mL MDH and 15 mM NADH). After 10 min, the solution was tested by a PGM.

### Lactate Assay

In a typical test in buffer, 80 μL HEPES buffer was spiked by 1 μL lithium L-lactate stock solution to a final concentration of 0-2 mM lactate to serve as test samples. Then 5 μL 160 U/mL lactate oxidase (LOx), 5 μL 800 U/mL LDH and 10 μL 100 mM NADH in HEPES buffer (200 mM HEPES pH 7.5, 50 mM KCl, 4 mM MgCl₂) were added to the above samples to initiate the coupled enzymatic reactions (final concentration of the reagents were 8 U/mL LOx, 40 U/mL LDH and 10 mM NADH). After a defined time (10, 20, 40, 60 or 80 min), the solution was tested by a PGM.

In a typical test in human serum, 15 μL human serum spiked with 0-16 mM lactate was mixed with 65 μL HEPES Buffer to make a test sample. The protocol for the test was then the same as that for the test in buffer shown above, except for a 20 min reaction time.

### Background Glucose Removal in G6PD Assay

Samples of 70 μL human serum spiked by 0-3 U/mL G6PD were added with 2.5 μL 256 mM MgCl2, 5 μL 320 mM ATP and 2.5 μL 100 U/mL hexokinase (final concentrations were 8 mM MgCl2, 20 mM ATP and 5 U/mL hexokinase). In 5 minutes, the background glucose in human serum was reduced to an undetectable level for PGM. Then 10 μL 600 mM glucose-6-phosphate and 10 μL 250 mM NADP+ in HEPES Buffer were added to the above samples to initiate the enzymatic reaction (final concentrations of the reagents were 60 mM glucose-6-phosphate and 25 mM NADP+). After 6 min, the solution was tested by a PGM.

### Ethanol Assay

In a typical test, 80 μL human serum was spiked by 1 μL ethanol stock solution to a final concentration of 0-1% ethanol as samples, and then 10 μl, 250 mM NAD and 10 μL 2.5 kU/mL alcohol dehydrogenase (ADH) in Buffer A (200 mM HEPES buffer pH 7.5, 4 mM MgCl2, 50 mM KCl) were added to the above samples (final concentration of 255 mM NAD₊ and 250 U/mL ADH) to initiate the enzymatic reaction (see below). After 10 minutes, the solution was tested by a PGM.

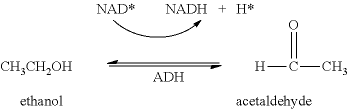

### Example 2

### Response of PGMs to NADH

This example describes results showing that PGMs can be used to measure changes in NADH concentration.

As shown in FIG. 2B, the glucose meter showed a linear response to the NADH concentration from 0.5 mM to 10 mM, which indicated NADH/NADPH is PGM-detectable. The reason is that the oxidative reagents on PGM strips (electrodes) are NADH responsive (NADH induces the oxidation reaction on the electrode), while NAD⁺/NADP⁺ is PGM-inert. Nicotinamide adenine dinucleotide phosphate (reduced form, NADPH) exhibited a very similar response due to its NADH-like chemical properties. However, NAD+/NADP+ did not give any signal in PGMs. In fact, NADH/NADPH acts similarly as the other cofactors used in the PGM strips, so PGMs can detect them.¹⁷⁻¹⁹

Taking advantage of the selective response of PGMs to NADH/NADPH over NAD+/NADP+, quantitative detection of NADH/NADPH concentration changes for the assays of various disease-related enzyme activities and metabolites can be achieved using PGMs, as shown in the Examples below. In these methods, NADH-dependent enzymes were used that can catalyze targets, such as lactate, into products and in the process convert NAD into NADH (or NADH to NAD⁺), which can then be detected by the PGM (FIG. 2A). In this way, a direct correlation between the presence and concentration of the target in the sample and the presence and intensity of the PGM signals is achieved. In the absence of the target, the redox reaction will not occur, generating no NADH, and thus no signal in PGM. NAD+-dependent enzymes can be applied to this PGM-based sensor design, as shown in FIG. 2C.

### Example 3

### Detection of Glucose-6-Phosphate Dehydrogenase (G6PD)

This example describes results showing PGMs can be used to measure G6PD by monitoring NADH or NADPH concentration.

G6PD deficiency is the most common defect of red blood cells, being present in more than 400 million people worldwide. In 2010, it resulted in about 4,000 deaths globally. As shown in FIG. 1A, G6PD is responsible for catalyzing the reaction of glucose-6-phosphate oxidation in the presence of NAD⁺/NADP⁺ to yield 6-phosphogluconolacetone and NADH/NADPH, converting NAD+ into NADH at the same time.¹¹ Therefore, by monitoring NADH concentration increase over time using a PGM, the activity of G6PD in samples can be quantified.

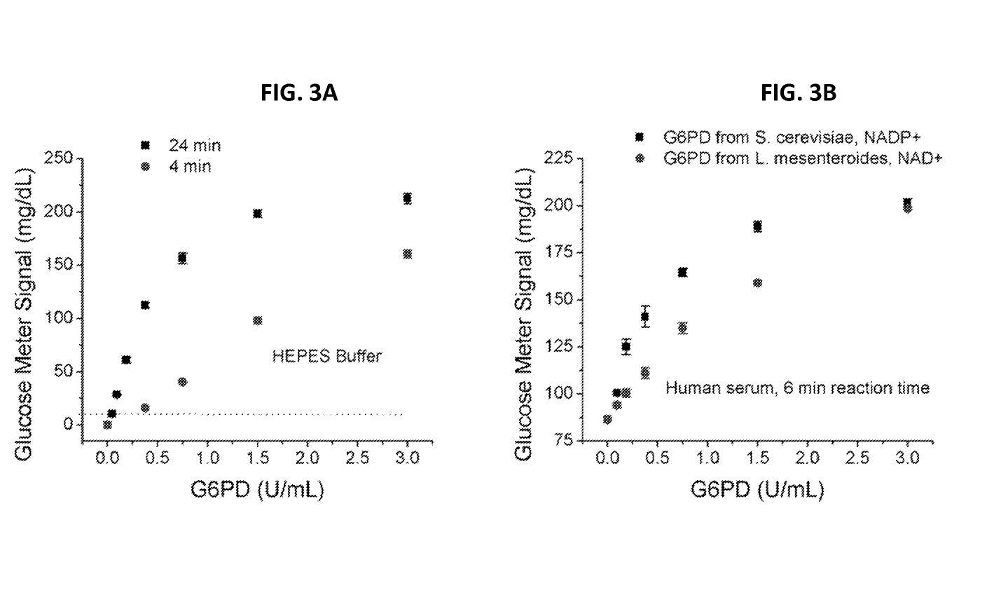

HEPES Buffer solutions spiked by different concentrations of G6PD from yeast (S. cerevisiae) were added with glucose-6-phosphate and NAD+. As shown in FIG. 3A, increasing PGM signals were observed for the samples containing increasing activities of G6PD after 4 or 24 minute reaction.

Another experiment was performed using both G6PDs from yeast and L. mesenteroides. The two types of G6PDs have different preferences for cofactors (yeast G6PD is specific to NADP+, while G6PD from L. mesenteroides can use both NADP+ and NAD+). As illustrated in FIG. 3B, the activities of both G6PDs were successfully quantified in human serum using PGM in 6 minutes, demonstrating the method can be used to detect G6PD from difference sources.

The method was capable of detecting G6PD as low as 0.07 U/mL, which is far below the clinic cut-off level of G6PD (around 1.2 U/mL, calculated from the cut-off level of around 8.3 U G6PD per gram hemoglobin and around 0.145 g hemoglobin per mL blood)^{10,11,16} for the diagnosis of G6PD deficiency.

### Example 4

### Detection of Pyruvate Kinase (PK)

This example describes results showing PGMs can be used to measure PK by monitoring decreases in NADH.

In addition to G6PD, the disclosed methods can monitor the activity of another enzyme PK, which catalyzes the transformation of phosphoenolpyruvate and ADP into pyruvate and ATP (FIG. 1B).¹¹ The product pyruvate can be further converted into lactate in the presence of lactate dehydrogenase (LDH) by consuming NADH, thus the PK activity can be measured using PGMs via monitoring the decrease of NADH concentration over time.

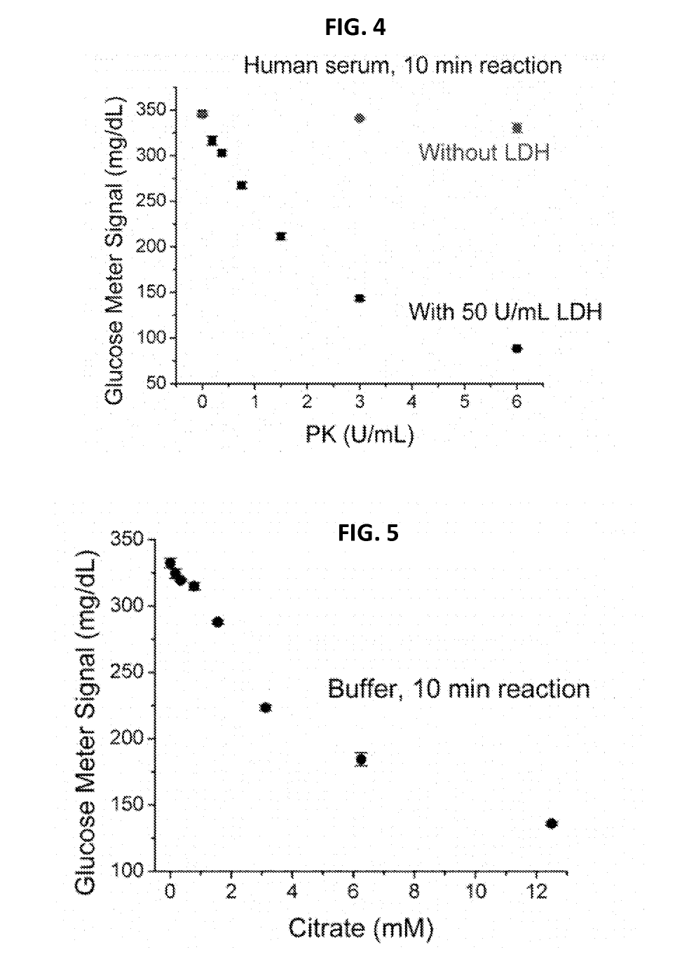

For human serum samples spiked with more PK activities, less PGM signals were observed (black dots in FIG. 4) after reacting with phosphoenolpyruvate, ADP, LDH and NADH for 10 minutes. To confirm the role of LDH in the second reaction, a control test was carried out by omitting LDH in the method. In this case, little decrease in PGM signals was observed regardless of the PK activity (red dots in FIG. 4), because the reaction stopped at the stage of pyruvate production and no NADH consumption occurred.

The method was sensitive to PK activity as low as 0.18 U/mL, which is also well below the clinic cut-off range of 2.2˜4.4 U/mL (calculated from the cut-off range of 15˜30 U PK per gram hemoglobin and around 0.145 g hemoglobin per mL blood)^{11,13,16} for the diagnosis of PK deficiency.

### Example 5

### Detection of Citrate

This example describes results showing PGMs can be used to measure citrate by monitoring NADH.

Besides enzyme activities, NADH was utilized as the link for PGMs to measure the concentrations of disease-related metabolites. Unlike enzymes, these metabolites are small organic molecules. They can serve as the substrates of enzymatic reactions in which NADH/NAD+ conversion is involved. The concentration of the metabolites is then converted to the concentration changes of NADH in samples, so that their detection can be achieved using PGMs. In fact, there are several metabolites involved in the above G6PD and PK assays, including glucose-6-phosphate, phosphoenolpyruvate and pyruvate. Their detection using PGMs can be simply realized by using the G6PD, PK and LDH as enzymes. For example, glucose-6-phosphate, phosphoenolpyruvate and pyruvate are substrates of G6PD, PK and LDH, respectively. They can be measured by adding the enzymes and display NADH increase (glucose-6-phosphate, NAD+ and G6PD), decrease (phosphoenolpyruvate, ADP, NADH, PK and LDH), and decrease (pyruvate, NADH and LDH), respectively.

Instead of them, the detection of citrate using NADH-dependent enzymatic reactions was demonstrated. Low citrate in human urine is an indication of high risk in kidney stone formation.⁵⁻⁷ Although there is no NADH-dependent enzyme utilizing citrate directly as substrate, a coupled enzymatic reaction (FIG. 1C) has been developed to convert citrate into oxaloacetate and then into NADH consumption by citrate lyase (CL) and malate dehydrogenase (MDH).¹⁴ Through this approach, citrate can be quantified by PGMs.

As shown in FIG. 5, the more citrate present in HEPES Buffer containing CL, MDH and NADH, the less PGM signal detected after a 10 minute reaction. The PGM signal decrease was due to the consumption of NADH by the coupled enzymatic reactions. The method was capable of detecting citrate concentration as low as 0.2 mM, which is lower than the normal cut-off concentration of citrate in human urine (1.75 mM, calculated by 500 mg per day in a daily urea volume of 1.5 L)^{5-7,14} for indicating the risk of kidney stone formation.

### Example 6

### Detection of Lactate Using Signal Amplification

This example describes results showing PGMs can be used to measure lactate by monitoring NADH.

Lactate is a marker for glycolysis and anaerobic metabolism in clinical diagnostics. High lactate concentration in blood is an indicator of tiredness and acidosis.⁸ During exercising or abnormal conditions, monitoring lactate concentration in blood is useful, for example to evaluate or determine the “lactate threshold”, which indicates the physical training level of a sportsman. In addition, lactic acidosis is also the most severe side effects of widely prescribed biguanide drugs for diabetes.⁹ Successful monitoring of lactate is thus helpful for monitoring the safety of the drugs.

Lactate dehydrogenase (LDH) can catalyze the inter-conversion between pyruvate/NADH and lactate/NAD+. However, LDH has a strong preference for lactate production (the right side of the equilibrium shown in FIGS. 1B and 1D), thus the conversion of lactate/NAD+ into pyruvate/NADH is inefficient even in the presence of high concentrations of lactate, NAD+ and LDH.¹² To efficiently convert lactate into the consumption of NADH, a signal amplification method was developed for lactate detection using PGMs based on the NADH-based approach.

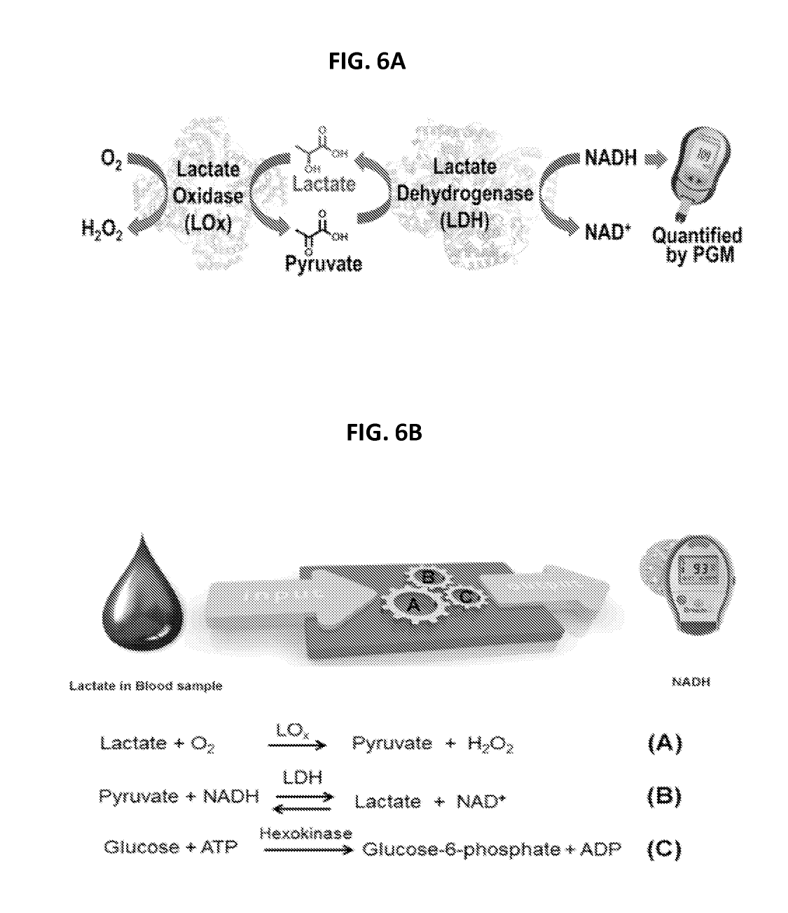

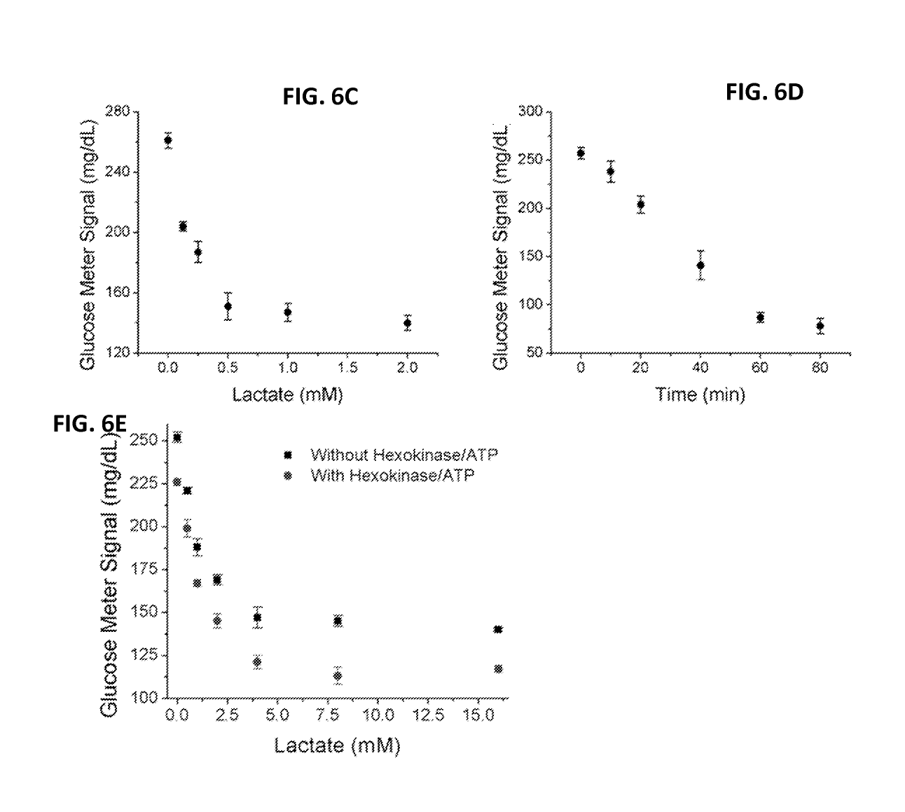

Provided herein is a homogeneous assay for the detection of lactate by a multi-enzymatic cascade reaction using PGM (FIGS. 6A and 6B). In the presence of lactate oxidase (LOₓ), the lactate in blood samples can be converted to pyruvate. At the same time, lactate dehydrogenase (LDH) catalyzes the conversion of pyruvate to lactate in the presence of NADH. These two reactions result in the consumption of NADH, which can be detected using PGM. As shown in FIG. 6B, hexokinase can be used to remove the background glucose in the blood samples (hexokinase converts glucose to glucose-6-phosphate (G6P), which is PGM-inert) (e.g., see Examples 7 and 8).

To induce sufficient NADH concentration changes in the presence of lactate, an amplified method was utilized via the coupled enzymatic reactions involving lactate oxidase (LOx) and LDH based on substrate recycling (FIG. 1D).^{31,32} In this approach, lactate was converted to pyruvate by lactate oxidase (LOx) in the presence of oxygen, and the product pyruvate was further converted back to lactate by LDH and consumed NADH. In total, lactate was recycled in the coupled enzymatic reaction and served like a “catalyst” to induce the consumption of NADH in multiple turnovers, enabling signal amplification.

As shown in FIG. 6C, in HEPES buffer containing 10 mM NADH, 8 U/mL LOx and 40 U/mL LDH, sensitive detection of lactate is achieved by measuring the decrease of NADH concentration in the samples. FIG. 6C shows the detection of lactate concentrations by allowing the mixture to react for 20 minutes before PGM measurement, while the FIG. 6D illustrates the time dependent NADH consumption for a sample containing 0.125 mM lactate, suggesting multiple turnovers (about 1 turnover per min for lactate) occurred for signal amplification.

The method had a detection limit of 0.01 mM lactate and a detection range of 0 to 2 mM (FIG. 6C), which is well capable of measuring the typical lactate concentrations in blood (5 to 20 mM),^{8,31,32} for example by dilution of samples (e.g., 5 to 10 or 5 to 100 fold dilution).

### Example 7

### Detection of Lactate in Human Serum

The method was further applied for quantitative lactate detection in human serum.

Human serum samples (spiked with different concentrations of lactate, 0 to 16 mM) were diluted 5-fold and then measured under the same condition as that in HEPES Buffer. In some samples, in order to remove the background glucose in human serum, 5 U/mL hexokinase and 5 mM ATP were added.

As shown in FIG. 6E, the detection range for lactate was 0 to 16.0 mM, with a detection limit of 0.3 mM. Thus, lactate can be detected using PGMs.

### Example 8

### Background Glucose Removal

This example describes results of measuring G6PD using commercially available PGMs following removal of blood glucose.

For some samples such as serum and blood, glucose originally present as background. In addition, the background glucose concentrations in different patient samples may vary and make the detections patient-dependent. Therefore, a method was developed to remove this background glucose to enhance the performance of the PGM-based method for monitoring enzyme activities and metabolites. With such a method, the patient-to-patient variations in our NADH-based method due to difference in blood glucose can be minimized. Fortunately, glucose differs from NADH, though both of them are detectable by PGMs. Hexokinase was used as the enzyme to remove glucose while maintaining NADH. Hexokinase catalyzes the conversion of glucose into glucose-6-phosphate in the presence of ATP.³⁰ Glucose-6-phosphate is not detectable in PGMs and ATP does not interfere with PGM measurement. Thus, this method is efficient in removing the background glucose.

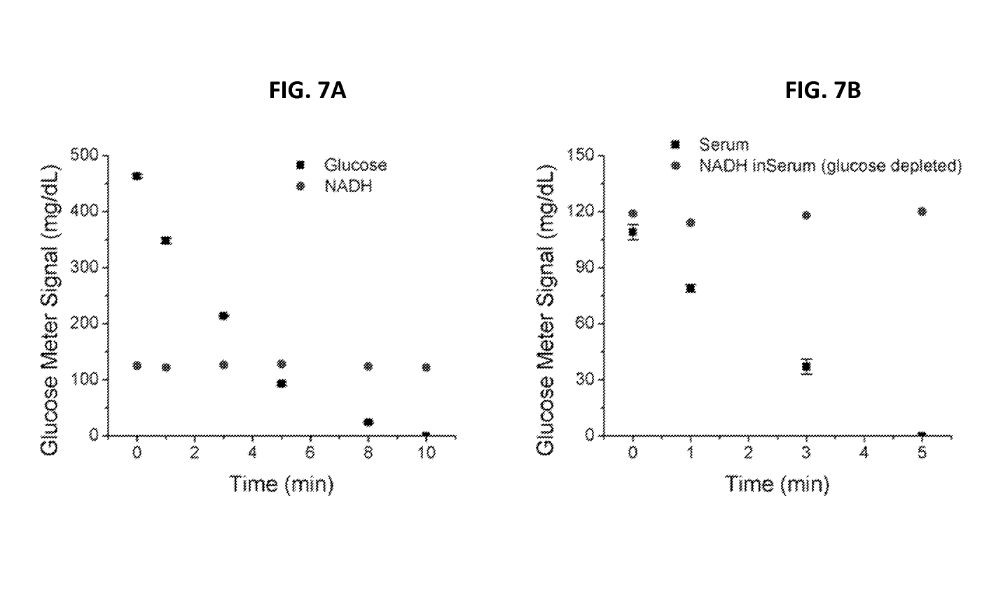

FIG. 7A shows the result of removing 20 mM glucose (much higher than blood glucose concentration) in HEPES Buffer (200 mM HEPES pH 7.5, 50 mM KCl, 4 mM MgCl₂) using 25 mM ATP and 5 U/mL hexokinase. Glucose (black squares) in the solutions was removed within 10 min to an undetectable level for PGMs. In contrast, the method did not affect the detection of NADH (red dots) by PGMs, so that the NADH-based detections of enzyme activities and metabolites can be carried out in the presence of hexokinase and ATP to remove background glucose signals. In addition to HEPES Buffer as the medium, this method was performed in human serum and the results are shown in FIG. 7B. Human serum samples were added with 8 mM MgCl₂ (essential for hexokinase activities), 25 mM ATP and 5 U/mL hexokinase. In 5 minutes, the background glucose in human serum was reduced to an undetectable level for PGMs. In contrast, NADH detection by PGMs was not affected in the presence of 25 mM ATP and 5 U/mL hexokinase.

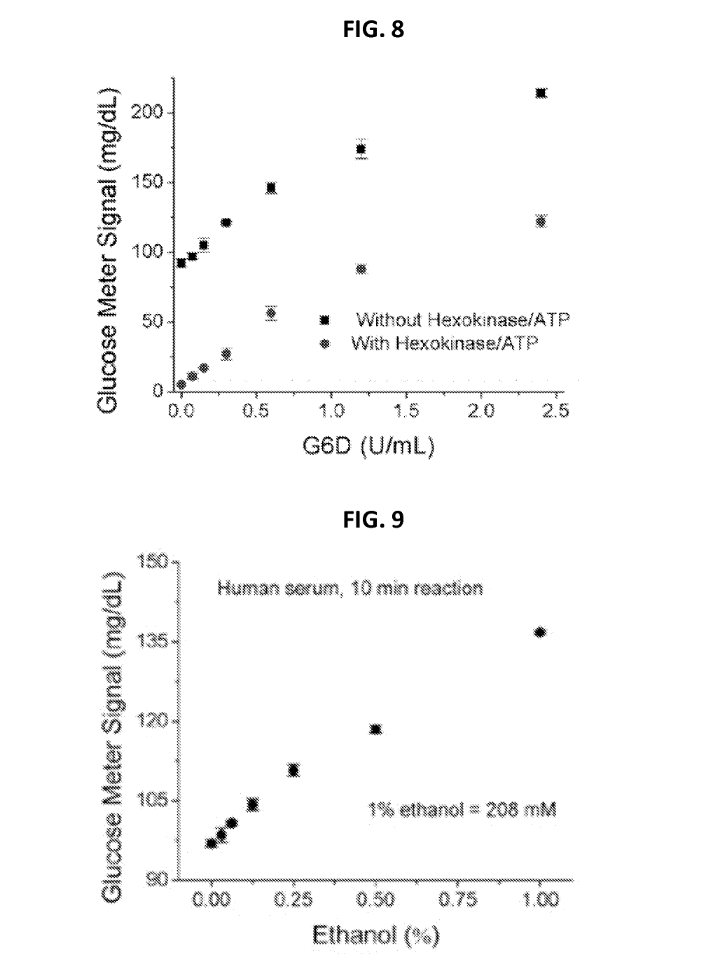

The success in background glucose removal was used to further apply the method to improve the performance of PGM-based detection of targets. Taking G6PD detection in human serum as an example, hexokinase and ATP were added to human serum samples containing different activities of G6PD to convert glucose into glucose-6-phosphate, and then introduced glucose-6-phosphate and NADP+ into the solutions to initiate the enzymatic reaction to produce NADPH for PGM measurement. Because glucose-6-phosphate was added in excess (>60 mM), the G6PD activity was found to be independent on the glucose-6-phosphate concentration increased by hexokinase under these conditions. As shown in FIG. 8, without the addition of hexokinase and ATP to remove glucose, a background signal about 88 mg/dL was present due to the original glucose in the human serum. In contrast, no background signal was observed when hexokinase and ATP were introduced to convert glucose into glucose-6-phosphate, suggesting the effective background glucose removal using the hexokinase-ATP method.

### Example 9

### Detection of Ethanol

This example describes results of measuring ethanol using commercially available PGMs.

As shown in FIG. 9, the more ethanol spiked in human serum, the more PGM signal is detected due to the production of NADH from ethanol. The method was able to quantify ethanol concentration as low as around 0.03%, which is the normal cut-off blood level.

### Example 10

### Variation of the Method

A modified lactate detection protocol was used for lactate detection in 100% human serum and in animal blood (calf whole blood).

The mixture of Reagent A (10 mM NADH, 8 U/mL LOx, 40 U/mL LDH, 50 mM ATP, 50 U/mL hexokinase) and Reagent B (0-5 mM lactate in HEPES buffer, 200 mM HEPES pH 7.5, 50 mM KCl, 4 mM MgCl₂) were directly applied to the sample (2 μL of Reagent B+8 μL of Reagent A), and lactate detected measured using PGM after 10 minute reaction. The total assay time using this protocol is 10 minutes, with a “Turnover rate” (NADH vs. lactate) of about 3.2/min. Thus, this modification can improve the time-to-result.

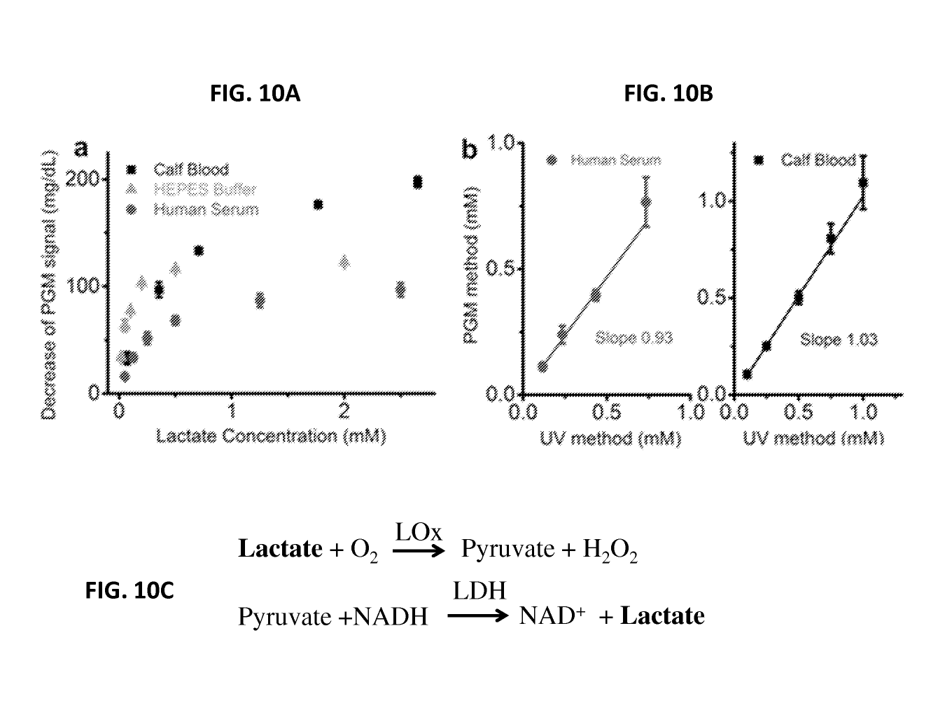

As shown in FIG. 10A, the NADH-PGM based method can be used to detect lactate in 10 minutes in HEPES buffer, 100% human serum, and calf blood samples, within sub-mM detection limits. For example, the detection range of lactate detection in HEPES buffer was 0 to 5 mM, with a detection limit of 0.034 mM.

Validation tests were also performed using an enzymatic UV-based method, demonstrating the ability to accurately detect lactate in human serum and calf blood (FIG. 10B) at sub-mM level. The design of enzymatic reaction for lactate detection using UV-vis method was shown in FIG. 10C. In the presence of lactate oxidase (LOx), the lactate in samples can be converted to pyruvate. At the same time, lactate dehydrogenase (LDH) catalyzes the conversion of pyruvate to lactate in the presence of NADH. These two reactions result in the consumption of NADH. The UV-vis absorption of NADH at 340 nm was then monitored using a spectrophotometer. The NADH consumption rate could be calculated from UV-vis absorption of NADH at 340 nm, which is directly related to the concentration of lactate in the samples.

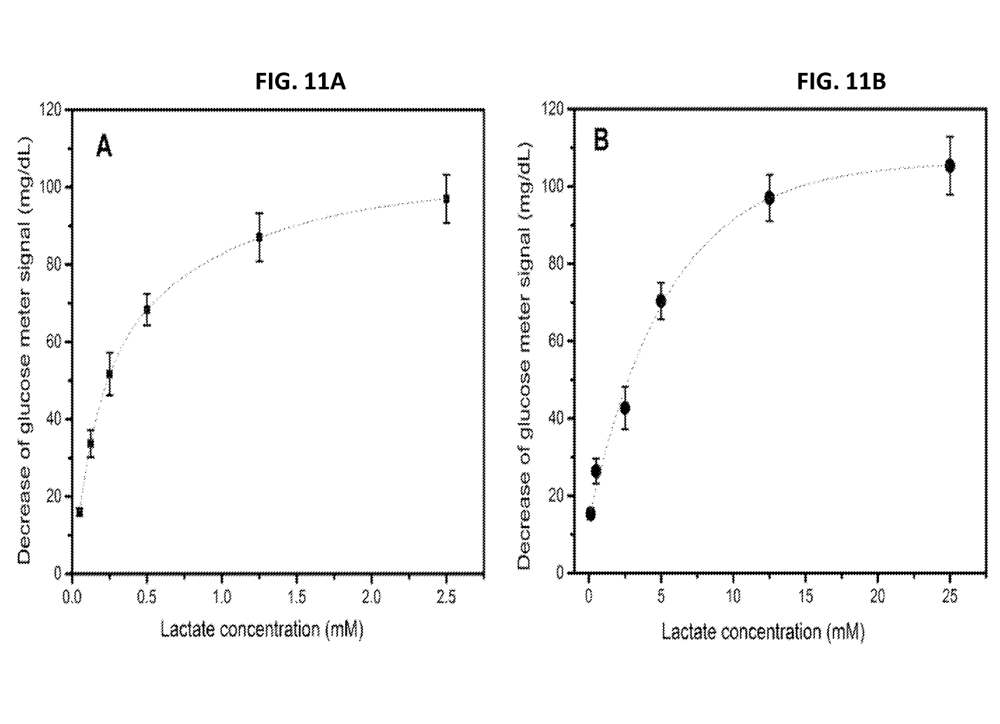

In practical applications, it is important to tune the dynamic range of an assay in order to meet different requirements at various circumstances. As shown in FIGS. 11A and 11B, the dynamic range for lactate detection in human serum could be tuned from 0 to 2.5 mM to 0 to 25.0 mM by simply altering the ratio between NADH and enzymes (LOₓ and LDH). In FIG. 11A, 10 μL 50 mM NADH was used, while in FIG. 11B 20 μL 50 mM NADH was used.

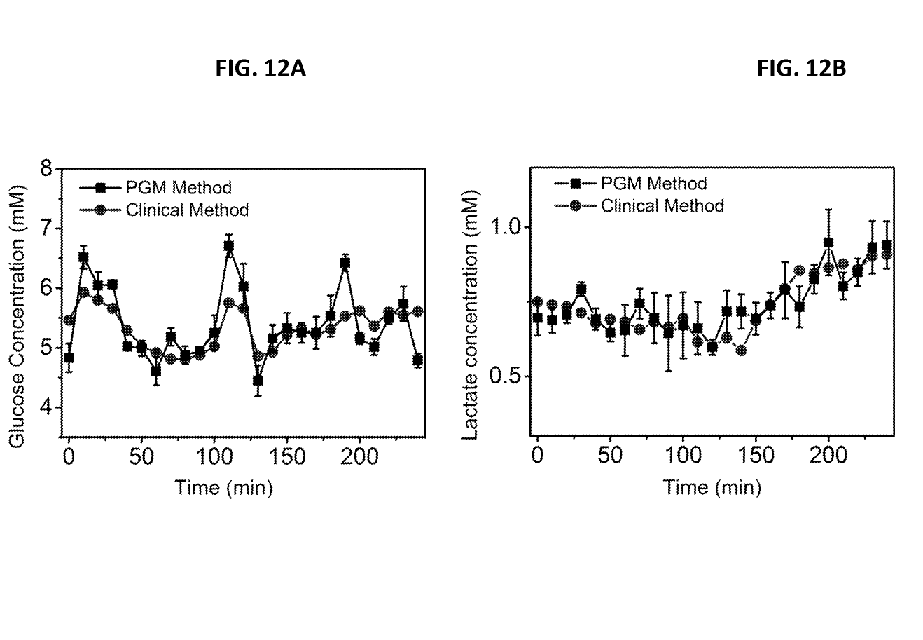

The NADH-PGM method was used to detect lactate in a sample in which glucose was removed. As shown in FIGS. 12A and 12B, a good correlation between the disclosed PGM-based method and the clinical lactate analyzer (YSI 2300 STAT Plus) was achieved. Thus, the background glucose in a blood sample can be removed, and the detection of lactate is independent of the glucose level in the sample.

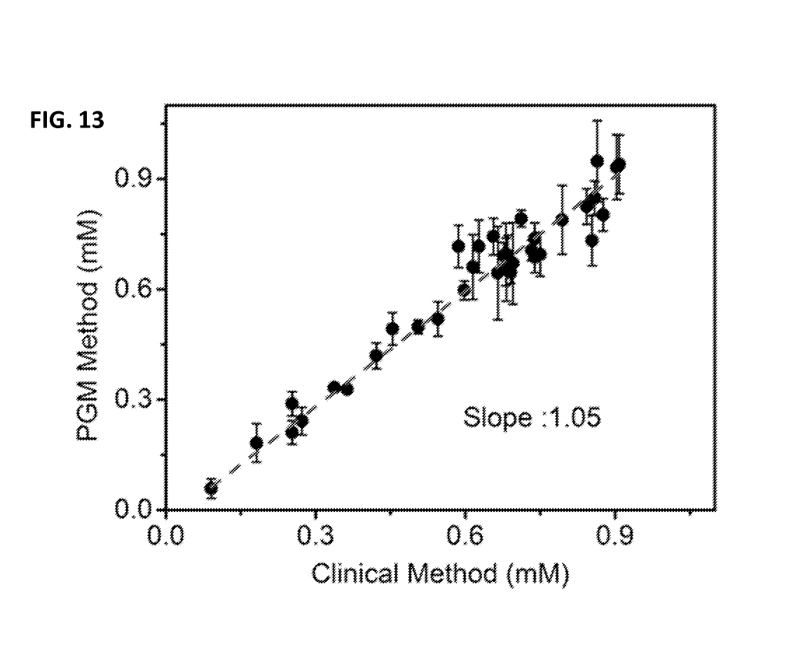

The NADH-PGM based lactate sensor method disclosed herein was compared to results achieved using clinical lactate analyzer (YSI 2300 STAT Plus). A series of human plasma samples with different lactate levels were evaluated. As shown in FIG. 13, a positive correlation between these two methods was found, with a slope of 1.05 and a correlation coefficient of 0.99, demonstrating that the results from the two methods matched within the experimental error.

In summary, the disclosed PGM-based method can be applied into lactate detection in various sample types, including buffer, 100% human serum, and animal blood. The total assay time can be <10 min, blood sample volume of about 2 μL, with high sensitivity and tunable dynamic range.

### Example 11

### Detection of 3-β-Hydroxybutyrate

This example describes methods that can use PGMs to measure 3-β-hydroxybutyrate (3HB) by monitoring NADH.

The American Diabetes Association advises that blood ketone testing methods that quantify 3-β-hydroxybutyrate (3HB) will be desirable for the diagnosing and monitoring ketoacidosis for diabetic patient management. Normal levels of 3HB are recognized below 1 mM while hyperketonemia is defined in the range from 1 to 3 mM, and ketoacidosis is defined above 3 mM. Various methods have been developed for the estimation of 3HB concentration, mostly based on the enzymatic dehydrogenation to acetoacetate by 3-β-hydroxybutyrate dehydrogenase (HBDH), followed by the detection of NADH. However, these methods are time consuming or require special apparatus, e.g., gas chromatography, and they are not suitable to be used for point-of-care detection of 3HB. This disclosure provides methods of using NADH-responsive PGMs to detect 3HB, by taking advantage of the NADH-dependent HBDH.

As shown in FIG. 14, the enzyme 3-hydroxybutyrate dehydrogenase (HBDH) catalyzes the production of acetoacetate and NADH from 3HB and NAD⁺. The produced NADH can be quantified using a PGM. To minimize the interference from blood glucose, hexokinase can be employed to convert glucose to G6P, which is PGM-inert (e.g., not detected by the PGM). Since the NAD⁺, 3HB, and acetoneacetate are all PGM-inert, the readout from PGM will be directly related to the amount of NADH produced from the enzymatic reaction. The results can be used to calculate the concentration of 3HB in blood (quantitatively or qualitatively).

### Example 12

### Exemplary Lateral Flow Strip

This example describes an exemplary lateral flow strip that can be used with the disclosed methods, in combination with PGMs to measure a target by monitoring NADH.

Interchangeable strips that incorporate the steps of the disclosed solution-based assays into lateral flow strips are provided. As shown in FIG. 15, the lateral flow strip includes a sample pad where the sample (e.g., blood) is applied. Through lateral flow, the sample containing target (e.g., lactate or 3HB) will move toward one or more reaction pads where the reaction reagents (e.g., ATP, hexokinase, LOx, LDH, and/or NADH) are immobilized. The presence of target will trigger the enzymatic reactions that can generate or consume NADH, which then will travel to an absorption pad where it will be measured by the PGM.

### Example 13

### Detection of Pyruvate

This example describes results showing PGMs can be used to measure pyruvate by monitoring NADH consumption. Pyruvate is a key intermediate in cellular metabolic pathways. It is the output of the metabolism of glucose (glycolysis).

To detect pyruvate, a homogeneous assay was developed using a multi-enzymatic cascade reaction using PGM. As shown in FIG. 16A, using lactate dehydrogenase (LDH), the pyruvate in a sample can be converted to lactate in the presence of NADH. At the same time, lactate oxidase (LOx), catalyzes the conversion of lactate back to pyruvate. These two reactions result in the continuously consumption of NADH, which can be detected by a PGM. To minimize the interference from blood glucose, hexokinase was employed to convert glucose to glucose-6-phosphate (G6P), which is PGM-inert.

In a typical test, 2 μL of pyruvate sample in Buffer A (200 mM HEPES buffer pH 7.5, 4 mM MgCl2, 50 mM KCl) was added to 8 μL of Reagent mixture (200 U/mL LDH, 50 mM NADH, and 50 U/mL LOx, 1:1:1) to initiate the enzymatic reaction (FIG. 16A). After 5 minutes, the solution was tested by a PGM.

As shown in FIGS. 16B and 16C, in the presence of LOx, pyruvate in the sample was detected at a range of 2.5 to 500 uM in 5 minutes, while in the absence of LOx, pyruvate in the sample was detected at a range of 0.5 to 50 mM.

### Example 14

### Detection of Adenosine Diphosphate (ADP)

This example describes results showing PGMs can be used to measure ADP by monitoring NADH consumption.

Adenosine diphosphate (ADP) plays a central role as the product of many metabolic reactions. Detection of ADP can therefore be used to monitor the progress of numerous reactions including ATPases that produce ADP and Pi and kinases that produce ADP and a phosphorylated product. ADP can be detected by a PGM by taking advantage of the coupled enzyme system of pyruvate kinase and lactate dehydrogenase.

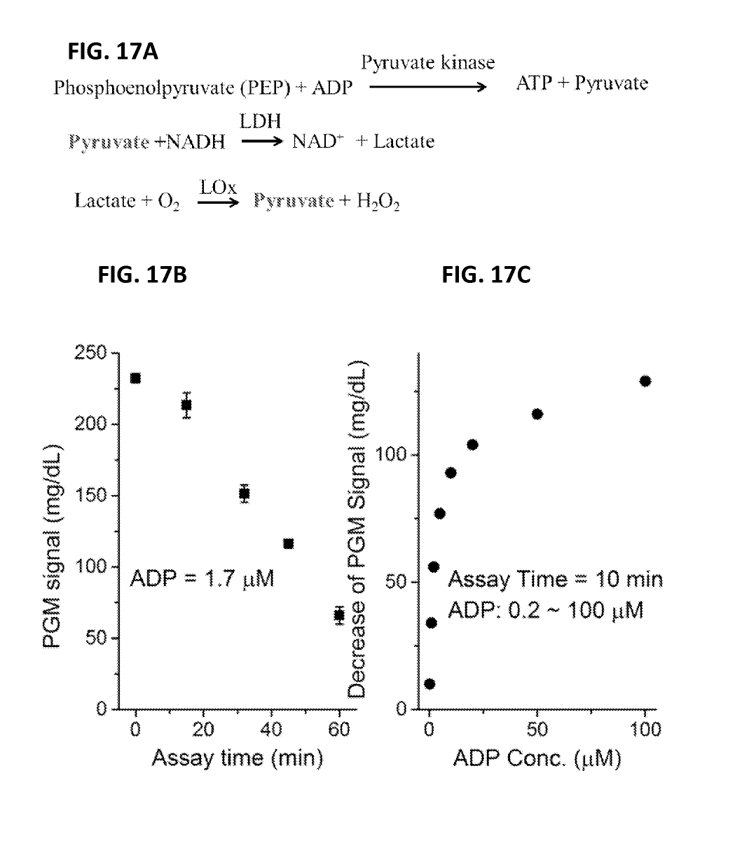

In a typical test, 2 μL of ADP sample in Buffer A (200 mM HEPES buffer pH 7.5, 4 mM MgCl2, 50 mM KCl) was added to 8 μL of Reagent mixture (50 mM PEP, 200 U/mL Pyruvate Kinase, 200 U/mL LDH, 50 mM NADH, and 50 U/mL LOx, 1:1:1:1:1) to initiate the enzymatic reaction (FIG. 17A). After 10 minutes, the solution was tested by a PGM.

As shown in FIG. 17A, the pyruvate kinase catalyzes the transfer of a phosphate group from phosphoenolpyruvate (PEP) to ADP, yielding one molecule of pyruvate and one molecule of ATP. With lactate dehydrogenase (LDH), the resulting pyruvate can be converted to lactate in the presence of NADH. At the same time, lactate oxidase (LOx), catalyzes the conversion of lactate back to pyruvate. These three reactions result in the continuously consumption of NADH, which is detected by PGM.

It was observed that the PGM signal decreases with the enzymatic reaction time in the presence of 1.7 μM ADP (FIG. 17B), and a detection range of 0.2 to 100 μM was observed with 10 minutes (FIG. 17C).

### Example 15

### Detection of Adenosine Triphosphate (ATP)

This example describes results showing PGMs can be used to measure ATP by monitoring NADH consumption.

Adenosine triphosphate (ATP) is an important substrate in living organisms. Being a major energy carrier of the cell, it plays a critical role in the regulation of cellular metabolism and biochemical pathways in cell physiology. It has also been used as an indicator for cell viability and cell injury. In addition, some diseases are related to ATP, such as angiocardiopathy. Therefore, the detection and quantification of ATP is useful in biochemistry and clinical diagnosis. ATP conversion to ADP is catalyzed by many different enzymes.

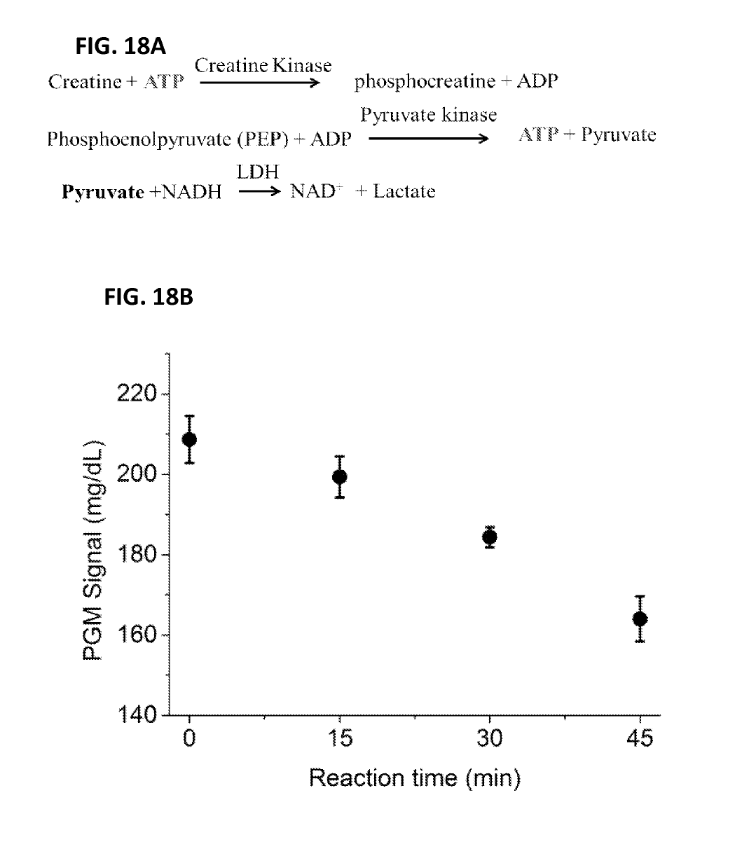

The disclosed methods were used to detect ATP using PGM. In a typical test, 2 μL of ATP sample in Buffer A (200 mM HEPES buffer pH 7.5, 4 mM MgCl2, 50 mM KCl) was added to 8 μL of Reagent mixture (50 mM Creatine, 2 kU/mL creatine kinase, 50 mM PEP, 200 U/mL Pyruvate Kinase, 200 U/mL LDH, 50 mM NADH, and 50 U/mL LOx, 1:1:1:1:1:1:1) to initiate the enzymatic reaction (FIG. 18A). After 10 minutes, the solution was tested by a PGM.

As shown in FIG. 18A, in the presence of creatine kinase, ATP can be converted to ADP. At the same time, the pyruvate kinase catalyzes the transfer of a phosphate group from phosphoenolpyruvate (PEP) to ADP, yielding one molecule of pyruvate and one molecule of ATP. With lactate dehydrogenase (LDH), the resulting pyruvate can be converted to lactate in the presence of NADH. These three reactions result in the continuous consumption of NADH, which is detected by PGM.

As shown in FIG. 18B, the PGM signal decreased with the enzymatic reaction time in the presence of 1.4 μM ATP, showing a good performance of with the method for ATP.

### Example 16

### Detection of Creatinine

This example describes results showing PGMs can be used to measure creatinine by monitoring NADH consumption.

Creatinine, a waste product of muscle catabolism, is used to assess renal function, monitor the course of renal disease, and adjust renal function dependent drug dosages. The disclosed methods can be used to detect creatinine by coupling enzymatic reactions that connect creatinine concentration with NADH, which can then be detected using PGM. In some examples, normal levels of creatinine for an adult male is about 0.6-1.2 mg/dL and for an adult female about 0.5-1.1 mg/dL, but it can vary with age.

As shown in FIG. 19A, in the presence of creatininase, creatinine can be hydrolyzed to creatine; creatine kinase catalyses the conversion of creatine and consumes adenosine triphosphate (ATP) to create phosphocreatine (PCr) and adenosine diphosphate (ADP). Subsequently, the pyruvate kinase catalyzes the transfer of a phosphate group from phosphoenolpyruvate (PEP) to ADP, yielding one molecule of pyruvate and one molecule of ATP. With lactate dehydrogenase (LDH), the resulting pyruvate can be converted to lactate in the presence of NADH. At the same time, lactate oxidase (LOx), catalyzes the conversion of lactate back to pyruvate. By combining these five cascade enzymatic reactions with the disclosed methods, NADH is continuously consumed, which can be detected by PGM.

The disclosed methods were used to detect creatinine using PGM. In a typical test, 2 μL of creatinine sample in Buffer A (200 mM HEPES buffer pH 7.5, 4 mM MgCl2, 50 mM KCl) was added to 8 μL of Reagent mixture (1 KU/mL Creatininase, 50 mM Creatine, 2 kU/mL creatine kinase, 50 mM ATP, 50 mM PEP, 200 U/mL Pyruvate Kinase, 200 U/mL LDH, 50 mM NADH, and 50 U/mL LOx, 1:1:1:1:1:1:1) to initiate the enzymatic reaction (FIG. 19A). After 10 minutes, the solution was tested by a PGM.

As shown in FIG. 19B, the PGM signal decreased with the enzymatic reaction time in the presence of creatinine, showing a good performance of with the method for creatinine.

### REFERENCES

(1) Nyhan, W. L.; Barshop, B.; Ozand, P. T. Atlas of Metabolic Diseases; 2nd ed.; Oxford University Press: New York, 2005.
(2) Beutler, E. New England Journal of Medicine 1991, 324, 169-174.
(3) Cappellini, M. D.; Fiorelli, G. Lancet 2008, 371, 64-74.
(4) Zanella et al., British Journal of Haematology 2005, 130, 11-25.
(5) Coe et al., New England Journal of Medicine 1992, 327, 1141-1152.
(6) Moe, O. W. Lancet 2006, 367, 333-344.
(7) Sheng et al., Proc. Natl. Acad. Sci. USA 2005, 102, 267-272.
(8) Oliva, P. B. American Journal of Medicine 1970, 48, 209-&.
(9) Bailey, C. J.; Turner, R. C. New England Journal of Medicine 1996, 334, 574-579.
(10) Motulsky, A. G.; Campbell-Kraut, I. M. Population genetics of glucose-6-phosphate dehydrogenase deficiency of the red cell; Grune and Stratton: New York, 1961.
(11) Beutler, E. Blood—the Journal of Hematology 1966, 28, 553-&.
(12) Howell, B. F.; McCune, S.; Schaffer, R. Clinical Chemistry 1979, 25, 269-272.
(13) Miwa et al., British Journal of Haematology 1979, 43, 275-286.
(14) Warty et al., Clinical Chemistry 1984, 30, 1231-1233.
(15) Beutler, E.; Baluda, M.; Donnell, G. E. J. Lab. Clin. Med. 1964, 64, 695-705.
(16) Beutler, E. Red cell metabolism: a manual of biochemical methods; 3rd ed.; Grune and Stratton: New York, 1984.
(17) Clark, L. C.; Lyons, C. Annals of the New York Academy of Sciences 1962, 102, 29-45.
(18) Heller, A.; Feldman, B. Chem Rev 2008, 108, 2482-2505.
(19) Montagnana et al., Clinica Chimica Acta 2009, 402, 7-13.
(20) Carroll et al., Diabetes Technology & Therapeutics 2007, 9, 158-164.
(21) Xiang, Y.; Lu, Y. Nature Chemistry 2011, 3, 697-703.
(22) Xiang, Y.; Lu, Y. Anal Chem 2012, 84, 1975-1980.
(23) Xiang, Y.; Lu, Y. Chemical Communications 2013, 49, 585-587.
(24) Xiang, Y.; Lu, Y. Anal Chem 2012, 84, 4174-4178.
(25) Su et al., Biosensors & Bioelectronics 2013, 45, 219-222.
(26) Xu et al., Chemical Communications 2012, 48, 10733-10735.
(27) Yan et al., Journal of the American Chemical Society 2013, 135, 3748-3751.
(28) Su et al., Chemical Communications 2012, 48, 6909-6911.
(29) Mohapatra, H.; Phillips, S. T. Chemical Communications 2013, 49, 6134-6136.
(30) Jang, J. C.; Leon, P.; Zhou, L.; Sheen, J. Plant Cell 1997, 9, 5-19.
(31) Scheller et al., Anal Chem 1985, 57, 1740-1743.
(32) Asouzu et al., Anal Chem 1990, 62, 708-712.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

1. A method for detecting a target enzyme or target metabolite that is part of an enzymatic reaction that consumes or generates a coenzyme, comprising:
contacting a test sample with one or more starting products needed for an enzymatic reaction that utilizes the target enzyme or target metabolite and consumes or generates the coenzyme;
allowing the enzymatic reaction to consume or generate the coenzyme;
detecting the coenzyme with a personal glucose meter (PGM); and
determining whether the target enzyme or target metabolite is present in the test sample by correlating an amount of the coenzyme detected.

2. The method of claim 1, wherein the coenzyme is:
nicotinamide adenine dinucleotide (NADH) and/or nicotinamide adenine dinucleotide phosphate (NADPH);
reduced form of flavin adenine dinucleotide (FADH₂); or
reduced form of flavin mononucleotide (FMNH₂).

3. The method of claim 1, wherein the coenzyme is NADH or NADPH and wherein:
the target enzyme comprises glucose-6-phosphate dehydrogenase (G6PD) and the one or more starting products comprise G6P and NAD⁺ or NADP⁺;
the target enzyme comprises pyruvate kinase (PK) and the one or more starting products comprise phosphoenolpyruvate (PEP), adenosine diphosphate (ADP), NADH, lactate dehydrogenase (LDH), and optionally lactate oxidase (LOx);
the target metabolite comprises citrate and the one or more starting products comprise citrate lyase (CL), acetyl coenzyme A, malate dehydrogenase (MDH), 2 NADH and lactate dehydrogenase (LDH);
the target metabolite comprises ethanol and the one or more starting products comprise alcohol dehydrogenase (ADH) and NAD⁺;
the target metabolite comprises lactate and the one or more starting products comprise lactate oxidase (LOx), LDH and NADH;
the target metabolite comprises 3-β-hydroxybutyrate (3HB) and the one or more starting products comprise 3-hydroxybutyrate dehydrogenase (HBDH), and NAD⁺;
the target metabolite comprises pyruvate and the one or more starting products comprise LDH, LOx, and NADH; or
the target metabolite comprises creatinine and the one or more starting products comprise creatininase, ATP, creatine kinase, PK, PEP, LDH, LOx, and NADH.

4. The method of claim 1, wherein the coenzyme is FADH₂ and wherein:
the target enzyme comprises FAD-dependent D-lactate dehydrogenase and the one or more starting products comprise FADH₂;
the target enzyme comprises FAD-dependent sarcosine oxidase and the one or more starting products comprise FAD; or
the target metabolite comprises FAD-dependent D-proline dehydrogenase and the one or more starting products comprise FAD.

5. The method of claim 1, wherein the coenzyme is FMNH₂ and wherein:
the target enzyme comprises FMN-dependent L-lactate dehydrogenase and the one or more starting products comprise FMNH₂.

6. The method of claim 1, wherein the test sample is a blood sample or fraction thereof or a urine sample.

7. The method of claim 1, wherein the test sample is a blood sample or fraction thereof, and wherein the method further comprises contacting the sample with hexokinase, MgCl₂, and ATP under conditions sufficient to remove glucose in the test sample.

8. The method of claim 1, wherein the test sample is obtained from a subject, and the method further comprises determining that the subject has a disease based on the target enzyme or target metabolite detected.

9. The method of claim 1, wherein the coenzyme is NADH or NADPH and:
the target enzyme comprises glucose-6-phosphate dehydrogenase (G6PD) and the disease comprises G6PD deficiency, wherein the subject is determined to have G6PD deficiency when an decrease in NADH or NADPH relative to a normal control is detected;
the target enzyme comprises pyruvate kinase (PK) and the disease comprises PK deficiency, wherein the subject is determined to have PK deficiency when an increase in NADH or NADPH relative to a normal control is detected;
the target metabolite comprises citrate and the disease comprises prostate cancer or kidney stones, wherein the subject is determined to have prostate cancer or kidney stones or increase risk of developing kidney stone when a decrease in NADH or NADPH relative to a normal control is detected;
the target metabolite comprises lactate and the disease comprises lactic acidosis, wherein the subject is determined to have lactic acidosis when a decrease in NADH or NADPH relative to a normal control is detected;
the target metabolite comprises ethanol and the disease comprises ethanol poisoning, wherein the subject is determined to have ethanol poisoning when an increase in NADH or NADPH relative to a normal control is detected;
the target metabolite comprises 3-β-hydroxybutyrate (3HB) and the disease comprises ketoacidosis, wherein the subject is determined to have ketoacidosis when an increase in NADH or NADPH relative to a normal control is detected;
the target metabolite comprises pyruvate and the disease comprises pyruvate kinase deficiency (PKD), wherein the subject is determined to have PKD when a decrease in NADH or NADPH relative to a normal control is detected; or
the target metabolite comprises creatinine and the disease comprises renal disease, wherein the subject is determined to have renal disease when a decrease in NADH or NADPH relative to a normal control is detected.

10. The method of claim 1, wherein the coenzyme is FADH₂ and:
the target enzyme comprises FAD-dependent D-lactate dehydrogenase and the disease comprises FADH₂, wherein the subject is determined to have D-lactate toxicity when a decrease in FADH₂ relative to a normal control is detected;
the target enzyme comprises FAD-dependent sarcosine oxidase and the disease comprises FAD, wherein the subject is determined to have sarcosinemia when an increase in FADH₂ relative to a normal control is detected; or
the target metabolite comprises FAD-dependent D-proline dehydrogenase and the disease comprises FAD, wherein the subject is determined to have hyperprolinemia when an increase in FADH₂ relative to a normal control is detected;

11. The method of claim 1, wherein the coenzyme is FMNH₂ and:
the target enzyme comprises FMN-dependent L-lactate dehydrogenase and the disease comprises FMNH₂, wherein the subject is determined to have increased levels of L-lactate when a decrease in FMNH₂ relative to a normal control is detected.

12. The method of claim 1, wherein the method further comprises detecting the target enzyme or target metabolite in a normal control sample, and comparing the target enzyme or target metabolite in the test sample and the normal control sample.

13. The method of claim 1, wherein the method further comprises comparing a detected value for the coenzyme to a reference value or range of values for the target enzyme or metabolite expected in a normal subject, to determine whether the target enzyme or metabolite in the sample is increased or decreased relative to a normal subject.

14. The method of claim 1, wherein the PGM uses a test strip comprising glucose oxidase or glucose dehydrogenase.

15. A sensor, comprising:
a solid support to which is attached:
one or more starting products needed for an enzymatic reaction that utilizes a target enzyme or target metabolite and consumes or generates a coenzyme, wherein the one or more starting products comprise one or more enzymes and one or more of ATP, ADP, MgCl₂, NAD⁺, and NADP⁺;
optionally a sample pad that transports the one or more starting products and a sample to reagent pad to initiate the enzymatic reactions; and
optionally an absorption pad that transports a cofactor to PGM for detection

16. The sensor of claim 15, wherein:
the target enzyme comprises glucose-6-phosphate dehydrogenase (G6PD) and the one or more starting products comprise G6P and NAD+ or NADP+;
the target enzyme comprises pyruvate kinase (PK) and the one or more starting products comprise phosphoenolpyruvate (PEP), adenosine diphosphate (ADP), NADH and lactate dehydrogenase (LDH);
the target metabolite comprises citrate and the one or more starting products comprise citrate lyase (CL), acetyl coenzyme A, malate dehydrogenase (MDH), NADH and lactate dehydrogenase (LDH);
the target metabolite comprises ethanol and the one or more starting products comprise alcohol dehydrogenase (ADH) and NAD⁺;
the target metabolite comprises lactate and the one or more starting products comprise lactate oxidase (LOx), LDH and NADH;
the target metabolite comprises lactate and the one or more starting products comprise lactate oxidase (LOx), LDH and NADH;
the target metabolite comprises 3-β-hydroxybutyrate (3HB) and the one or more starting products comprise 3-hydroxybutyrate dehydrogenase (HBDH), and NAD⁺;
the target metabolite comprises pyruvate and the one or more starting products comprise LDH, LOx, and NADH; or
the target metabolite comprises creatinine and the one or more starting products comprise creatininase, ATP, creatine kinase, PK, PEP, LDH, LOx, and NADH;
the target enzyme comprises FAD-dependent D-lactate dehydrogenase and the one or more starting products comprise FADH₂;
the target enzyme comprises FAD-dependent sarcosine oxidase and the one or more starting products comprise FAD; or
the target metabolite comprises FAD-dependent D-proline dehydrogenase and the one or more starting products comprise FAD; or
the target enzyme comprises FMN-dependent L-lactate dehydrogenase and the one or more starting products comprise FMNH₂.

17. The sensor of claim 15, wherein the solid support comprises a membrane, a chamber of a fluidic device, or a bead.

18. A lateral flow device, a microfluidic device, or a macrofluidic device comprising:
the sensor of claim 15.

19. A microfluidic device comprising:
a sample entry port;
one or more mixing chambers containing one or more starting products needed for an enzymatic reaction that utilizes a target enzyme or target metabolite and consumes or generates a coenzyme;
optionally a filter; and
an exit port.

20. A kit comprising:
one or more sensors of claim 15; and
one or more of a buffer, a chart for correlating detected coenzyme level and amount of target enzyme or target metabolite present.

21. A method for detecting a target enzyme or target metabolite, comprising:
contacting one or more sensors of claim 15, with a test sample under conditions sufficient to allow the target enzyme or target metabolite in the test sample to interact with the one or more starting products, under conditions wherein a coenzyme will be generated or consumed;
detecting the coenzyme with a PGM; and
correlating the amount of target agent present in the sample to the amount of coenzyme detected.

22. A method for detecting a target agent, comprising:
introducing a test sample into the sample entry port of the microfluidic device of claim 19;
allowing a target in the test sample to interact with the holing chamber containing the one or more starting products;
allowing the coenzyme to be generated or consumed in one of the one or more mixing chambers;
detecting the coenzyme with a PGM; and
correlating the amount of target agent present in the sample to the amount of coenzyme detected.
